SEQUENCE LISTING (5 sequences)

SEQ ID NO: 1 - DNA, 764 bp, Homo sapiens
gcgggaccggggccgctggggtctggacgggggtcgccatgatccgctttatcctcatccagaaccgggcaggcaagacgcgcctggccaagtggtacatgcagtttgatgatgatgagaaacagaagctgatcgaggaggtgcatgccgtggtcaccgtccgagacgccaaacacaccaactttgtggagttccggaactttaagatcatttaccgccgctatgctggcctctacttctgcatctgtgtggatgtcaatgacaacaacctggcttacctggaggccattcacaacttcgtggaggtcttaaacgaatatttccacaatgtctgtgaactggacctggtgttcaacttctacaaggtttacacggtcgtggacgagatgttcctggctggcgaaatccgagagaccagccagacgaaggtgctgaaacagctgctgatgctacagtccctggagtgagggcaggcgagccccaccccggccccggcccctcctggactcgcctgctcgcttccccttcccaggcccgtggccaacccagcagtccttccctcagctgcctaggaggaagggacccagctgggtctgggccacaagggaggagactgcaccccactgcctctgggccctggctgtgggcagaggccaccgtgtgtgtcccgagtaaccgtgccgttgtcgtgtgatgccataagcgtctgtgcgtggagtccccaataaacctgtggtcctgcctggaaaaaaaaaaaaaaaaaa

SEQ ID NO: 2 - DNA, 1889 bp, Homo sapiens
gcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataa

SEQ ID NO: 3 - DNA, 4536 bp, Homo sapiens
aggctgtcactcaggtggcagcggcagaggccgggctgagacgtggccaggggaacacggctggctgtccaggccgtcggggcggcagtagggtccctagcacgtccttgccttcttgggagctccaagcggcgggagaggcaggcgtcagtggctgcgcctccatgcctgcgcgcggggcgggacgctgatggagcgcgccatcagcccggggctgctggtacgggcgctgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcaatgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttcgctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaa

SEQ ID NO: 4 - DNA, 4261 bp, Homo sapiens
agcttctggaaggcgctgctcttccggttctctgtcccggttcctggggttgcacagacagaccctgtaaacatgtcagggttcagtccggaactcatcgactacttggaagggaaaatctcctttgaggagttcgaacggcggagagaagagagaaaaacccgcgagaagaaaagtcttcaggaaaaaggcaagttatcagctgaagaaaatcccgatgactctgaagttccatcatcatcaggaattaactctaccaaatcccaagacaaagatgtcaatgaaggagaaacatcagatggagtgaggaagtcagttcacaaggtctttgcttccatgcttggagagaatgaagatgatgaggaggaagaggaagaagaggaggaggaggaggaggaggaagaaacacctgagcaacccactgcgggcgatgtatttgtattggagatggttctcaatcgtgaaaccaagaaaatgatgaaagagaaaaggcctcggagtaaacttcccagagctctgagaggtctcatgggtgaagccaacattcgttttgctcgaggagaacgtgaagaggcgatattgatgtgcatggaaatcataagacaagctcctctggcttatgagccattctctactctagccatgatatatgaggaccaaggtgacatggaaaaatcattgcagtttgagttgattgctgcgcatttaaatcccagtgacacagaagaatgggttagactggcagaaatgtctctggaacaagacaatattaagcaggctattttttgctatacaaaagctcttaaatatgaacctactaatgtccgttatctgtgggagcgatcaagcctttatgaacagatgggtgatcataaaatggccatggatggttataggcgtattttaaaccttttgtctccatctgatggcgaacgttttatgcagctggctagagatatggcaaagagttactatgaagccaatgatgttacttctgctattaacataattgatgaagctttctcaaaacaccagggcctagtctccatggaagatgttaacatagcagctgaactatatatttctaacaaacagtatgacaaagctttggagataattacagatttttctggaattgtgctggaaaaaaaaacttcagaagaaggcacctcagaagagaataaagctcctgagaatgttacctgcactatacctgatggcgtgccaatagatatcacagtgaagttgatggtctgccttgtacatctcaacattcttgaaccacttaatcctctcttgacaacactagtagaacagaatcctgaagatatgggagacctatacctagatgttgctgaagcttttctggatgttggtgaatataattctgcacttcccctcctcagtgctcttgtttgctctgaaagatacaaccttgcagtagtttggcttcgtcatgcagaatgtttaaaggccttaggctatatggagcgagctgctgaaagctatggcaaggtggttgatctggccccactccatttggatgcaaggatttcactttctacccttcagcagcagctgggccagcctgagaaagctctggaagctctggaaccaatgtatgatccagatactttagcacaggatgcaaatgctgcacagcaggaactgaagttattgcttcatcgttctactctgttgttttcacaaggcaaaatgtatggttatgtggataccttacttactatgttagccatgcttttaaaggtagcaatgaatcgagcccaagtttgtttgatatccagttccaagtctggagagaggcatctttatcttattaaagtatcgagagacaaaatatcagacagcaatgaccaagagtcagcaaattgtgatgcaaaagcaatatttgctgtgctcacaagcgtcttgacaaaggatgactggtggaatcttctgttgaaggccatatactccttatgtgacctatcccgatttcaagaggctgagttgcttgtagattcctcattggaatattactcattttatgatgacaggcaaaaacgcaaagaactagaatactttggtctgtctgctgcaattctggacaaaaatttcagaaaggcatacaactatatcaggataatggtaatggaaaatgtcaataaaccccagctctggaacattttcaatcaagttaccatgcactcccaagatgtacgacatcatcgcttctgtctccgtttgatgctgaaaaacccagaaaatcatgccctatgtgtcttaaatggacacaatgcatttgtatctggtagttttaagcatgcgcttggacagtatgtgcaagcctttcgcactcaccctgacgaacctctctatagcttctgtataggcctaacctttattcatatggcatctcagaagtatgtgttacggagacatgctcttattgtacagggcttttcctttcttaatcgatacctcagtttacgtgggccctgccaggaatcattctacaatttgggccgtggccttcatcagttggggctgattcatcttgcaatccactattatcagaaggccctggagctccctccacttgtggtagagggtatagaacttgaccagttagacttacgaagagatattgcctacaacttgtctctcatctatcagagcagtgggaataccggaatggctcaaacgcttttgtatacctattgttctatataaagcaccgcaactgagaacagagcaatggcagctgctgtgtgaggaccagtgtcttctgtctcagggcttattatttgtaactccaaaatagaaatgacaatttcagaattacctaacaaacagtgtatttatttttaatatgtgataatgatcttgtggtatatatgcaaaattattcctacaaaaatttgtatattggtctgtcattttcctttcacattctatagtgaattgttcccaatgttgaaatggacgtgtaagcctttgagctagcttggagtcgaatacactatttttcactcacaccatttattcatctttgtatttaatactatagctctgtcaatatcacatgaggcagtttttcaaatacgtataaacagaggttgcttattattaaaggaaagacaaagtgggactctttatgatgtcatgaccatgataactaagcacctaagaaaattatttaaaatagttatgtggtaggcagaaagacaaataatttagttttttacttttcaccagcatgtatcttagctacctaaactgaaacatgggaggctgggcttaattcaaaatatattgctccaaggcaaataaaaaaatgctttatctatatttgtggctttctgatgaaaaaatagagaagagcttgttcaataacaggacatggtttccatttcaagatcacaagtaatataagactgggcaagtagtacgtatggaataaaggacatactgctgattgataaagtaaaaaactttttttttgtttgtttactcatctccactatttattatatgttcttgaatttaagttaacagtactttttagatgatatactgttagcttaataacaactttttagggaaaaataaatgctgtaattaatgtgcacatgggttagtaacacccagcccaattgtgggagggaaacaagtagaggcttaggatcaaagaaataaaattgggacttattagaaattcttaccactgtttctactgtacacaaaactttctagttgagcagaatttgtatgcaataagtaaatatattgtatactccatgtgtataatttaaatgcattttatttttataattgaggttaactgtttcacatgcttaatttttactttatgccatttataggtaatggtagaggtaactgagatacagtaataagttagacttgtgtgttggaattctgtggaactgagcattctgtgctccgagtttctctcttaaattagctcactggactgtggctccagtgtctactaaatagccgtggaggaaataagtctccctaagggaaaaaaaaaccagctgatattgtatagttttgtttagcttttaaaatcatagcttccatttgctgagtttttactctgctactgtatgctaagtatggtgctaaacactttggatatgttatctctgattctctcagggaactgcaaggtagttattattgatattgtcatcaccactttgtatctagagattaaatagcatgttcaagatca

SEQ ID NO: 5 - DNA, 4248 bp, Homo sapiens
aaaggtaaccactcctttgtggaggtcgccagggtcattgtcgtggatttgcacagtcggctgggcggtgcaatggcggaaagaaaaggaacagccaaagtggactttttgaagaagattgagaaagaaatccaacagaaatgggatactgagagagtgtttgaggtcaatgcatctaatttagagaaacagaccagcaagggcaagtattttgtaaccttcccatatccatatatgaatggacgccttcatttgggacacacgttttctttatccaaatgtgagtttgctgtagggtaccagcgattgaaaggaaaatgttgtctgtttccctttggcctgcactgtactggaatgcctattaaggcatgtgctgataagttgaaaagagaaatagagctgtatggttgcccccctgattttccagatgaagaagaggaagaggaagaaaccagtgttaaaacagaagatataataattaaggataaagctaaaggaaaaaagagtaaagctgctgctaaagctggatcttctaaataccagtggggcattatgaaatcccttggcctgtctgatgaagagatagtaaaattttctgaagcagaacattggcttgattatttcccgccactggctattcaggatttaaaaagaatgggtttgaaggtagactggcgtcgttccttcatcaccactgatgttaatccttactatgattcatttgtcagatggcaatttttaacattaagagaaagaaacaaaattaaatttgggaagcggtatacaatttactctccgaaagatggacagccttgcatggatcatgatagacaaactggagagggtgttggacctcaggaatatactttactcaaattgaaggcgcttgagccatacccatctaaattaagtggcctgaaaggtaaaaatattttcttggtggctgctactctcagacctgagaccatgtttgggcagacaaattgttgggttcgtcctgatatgaagtacattggatttgagacggtgaatggtgatatattcatctgtacccaaaaagcagccaggaatatgtcataccagggctttaccaaagacaatggcgtggtgcctgttgttaaggaattaatgggggaggaaattcttggtgcatcactttctgcacctttaacatcatacaaggtgatctatgttctcccaatgctaactattaaggaggataaaggcactggtgtggttacaagtgttccttccgactcccctgatgatattgctgccctcagagacttgaagaaaaagcaagccttacgagcaaaatatggaattagagatgacatggtcttgccatttgagccggtgccagtcattgaaatcccaggttttggaaatctttctgctgtaaccatttgtgatgagttgaaaattcagagccagaatgaccgggaaaaacttgcagaagcaaaggagaagatatatctaaaaggattttatgagggtatcatgttggtggatggatttaaaggacagaaggttcaagatgtaaagaagactattcagaaaaagatgattgacgctggagatgcacttatttacatggaaccagagaaacaagtgatgtccaggtcgtcagatgaatgtgttgtggctctgtgtgaccagtggtacttggattatggagaagagaattggaagaaacagacatctcagtgcttgaagaacctggaaacattctgtgaggagaccaggaggaattttgaagccaccttaggttggctacaagaacatgcttgctcaagaacttatggtctaggcactcacctgccttgggatgagcagtggctgattgaatcactttctgactccactatttacatggcattttacacagttgcacacctattgcaggggggtaacttgcatggacaggcagagtctccgctgggcattagaccgcaacagatgaccaaggaagtttgggattatgttttcttcaaggaggctccatttcctaagactcagattgcaaaggaaaaattagatcagttaaagcaggagtttgaattctggtatcctgttgatcttcgcgtctctggcaaggatcttgttccaaatcatctttcatattacctttataatcatgtggctatgtggccggaacaaagtgataaatggcctacagctgtgagagcaaatggacatctcctcctgaactctgagaagatgtcaaaatccacaggcaacttcctcactttgacccaagctattgacaaattttcagcagatggaatgcgtttggctctggctgatgctggtgacactgtagaagatgccaactttgtggaagccatggcagatgcaggtattctccgtctgtacacctgggtagagtgggtgaaagaaatggttgccaactgggacagcctaagaagtggtcctgccagcactttcaatgatagagtttttgccagtgaattgaatgcaggaattataaaaacagatcaaaactatgaaaagatgatgtttaaagaagctttgaaaacagggttttttgagtttcaggccgcaaaagataagtaccgtgaattggctgtggaagggatgcacagagaacttgtgttccggtttattgaagttcagacacttctcctcgctccattctgtccacatttgtgtgagcacatctggacactcctgggaaagcctgactcaattatgaatgcttcatggcctgtggcaggtcctgtggatgaagttttaatacactcctcacagtatcttatggaagtaacacatgaccttagactacgactcaagaactatatgatgccagctaaagggaagaagaccgacaaacaacccctgcagaagccctcacattgcaccatctatgtggcaaagaactatccaccttggcaacataccaccctgtctgttctacgtaaacactttgaggccaataacggaaaactgcctgacaacaaagtcattgctagtgaactaggcagtatgccagaactgaagaaatacatgaagaaagtcatgccatttgttgccatgattaaggaaaatctggagaagatggggcctcgtattctggatttgcaattagaatttgatgaaaaggctgtgcttatggagaatatagtctatctgactaattcgcttgagctagaacacatagaagtcaagtttgcctccgaagcagaagataaaatcagggaagactgctgtcctgggaaaccacttaatgtttttagaatagaacctggtgtgtccgtttctctggtgaatccccagccatccaatggccacttctcaaccaaaattgaaatcaagcaaggagataactgtgattccataatcaggcgtttaatgaaaatgaatcgaggaattaaagacctttccaaagtgaaactgatgagatttgatgatccactgttggggcctcgacgagttcctgtcctgggaaaggagtacaccgagaagacccccatttctgagcatgctgttttcaatgtggacctcatgagcaagaaaattcatctgactgagaatgggataagggtggatattggcgatacaataatctatctggttcattaaactcatgcacattggagatttatcctggtttcttaggaatactactactctgattgtgtctactgattggctatcagaaccttaggctggacctaaatagattgatttcatttctaaccatccaattctgcatgtattcataattctatcaagtcatctttgattcctggacctaataaattttttttccctttctttgggtgtccaagagaaatggtttttgccaaactctttttaaaaaacaaattgttgctatttcctagaagtttctggtttttaagatgaacataaaagtgtcagtatgcttccttttatgaggtgtactttatactttgatgaaggctaaggtgtacctaacagctttttatagtatattcatttatggagttagctgtattttttttaaaaaaagaaaagtagtaaattagcagctttttttttttttaaatgaagagaacttcatgatgatttaaaagaactaatgagaatattttgacttgacttcaattactaagtattttgtataaagaacaaataggtcctgcattttataagactttttaaagctttaattattcaactaagaaccaataatggcttcttcaaggctaatatcaatagtttataagcagtatatttcaccta

(19) United States
(12) Patent Application Publication
(10) Pub. No.: US 2023/0279502 A1
(43) Pub. Date: Sep. 7, 2023

(54) IDENTIFICATION OF ESTROGEN RECEPTOR POSITIVE (ER+) BREAST CANCERS THAT WILL NOT DEVELOP TAMOXIFEN RESISTANCE

(71) Applicant: Rutgers, The State University of New Jersey, New Brunswick, NJ (US)

(72) Inventors: Antonina Mitrofanova, New York, NY (US); Sarra M. Rahem, Nutley City, NJ (US)

(73) Assignee: Rutgers, The State University of New Jersey, New Brunswick, NJ (US)

(21) Appl. No.: 18/013,368

(22) PCT Filed: Jun. 29, 2021
(86) PCT No.: PCT/US21/39637

§ 371 Date: Dec. 28, 2022

Related U.S. Application Data
(60) Provisional application No. 63/045,878, filed on Jun. 30, 2020.

(51) Int. Cl. C12Q 1/6886

(52) U.S. Cl. CPC C12Q 1/6886; C12Q 2600/106; C12Q 2600/158

(57) ABSTRACT

Disclosed herein are methods of treating and identifying subjects with ER+ breast cancer that will or will not develop resistance of tamoxifen. In some examples such methods include measuring expression of adaptor-related protein complex 2, sigma-1 subunit (AP2S1) from the retrograde neurotrophin signaling pathway, cyclin-dependent kinase 2 (CDC2) from the loss of NLP from mitotic centrosomes pathway, general transcription factor IIIC subunit 3 (GTF3C3) from the RNA polymerase III transcription initiation from Type 2 promoter pathway, eukaryotic translation initiation factor 2-alpha kinase 3 (EIFA2AK3) from the EIF2 pathway, and leucyl-tRNA synthetase (LARS) from the valine, leucine and isoleucine biosynthesis pathway.

### CROSS REFERENCE TO RELATED APPLICATION

This application claims priority to U.S. Provisional application No. 63/045,878 filed on Jun. 30, 2020, herein incorporated by reference in its entirety.

### FIELD

This disclosure relates to methods of treating and identifying subjects with an estrogen receptor positive (ER+) breast cancer, that will not develop resistance (i.e., become resistant) or that will develop resistance, to tamoxifen chemotherapy treatment.

### BACKGROUND

Despite recent advances in diagnosis, classification, and therapeutic management, breast cancer (BC) remains one of the leading causes of cancer-related death in women worldwide.¹⁻³ Nearly 70% of all diagnosed cases of breast tumors are estrogen receptor-positive (ER+),⁴⁵ making treatments with anti-estrogen effects in the breast cells, such as tamoxifen, the standard-of-care for patients with ER+ breast cancers.^{4,6-9} Despite the significant success of tamoxifen administration, nearly 30% of treated patients develop therapeutic resistance, ultimately leading to metastasis and lethality.^{1,10} Therefore, prioritization of patients based on the risk of resistance to tamoxifen before treatment administration could play a significant role in personalize therapeutic planning for patients with ER+ breast cancer and builds a foundation to improve disease course and outcomes.

Tamoxifen is a selective estrogen receptor modulator (SERM) and has agonist or antagonist activity depending on the tissue type.¹¹ In the breast cells, tamoxifen directly binds to the ER, blocking estrogen from attaching to the receptor and thus inhibiting the activity of estrogen-regulated genes and causing the repression of estrogenic effects.^{4,5,12,13} However, the emergence of alternative mechanisms of estrogenic stimulation has been shown to cause emergence of resistance to tamoxifen. For example, some studies have demonstrated that ER+ breast cancers that overexpress HER2 and EGFR can activate the components of downstream signaling pathways which then stimulate both ER and estrogen receptor co-activator AIB1, and thus induce the estrogen agonistic activity of tamoxifen in breast cancer cells.^{14,15} Another study noticed that the increased expression of HER2 signaling can also downregulate progesterone receptor (PR) levels in the ER+ breast tumors, where losing the PR expression serves as a biomarker of hyperactive growth factor signaling, leading to another possible mechanism of tamoxifen resistance.¹⁶ Despite the emerging role of HER2 in tamoxifen resistance, it only accounts for 10% of ER+ breast cancers^{12,17} indicating more complex resistance mechanisms in these cases, presenting a central clinical problem for patients with ER+ breast cancer.^{4,5,10,12}

In recent years, several groups have developed gene expression signatures of tamoxifen response for ER+ patients, including 10 gene-signature by Men et al.,¹⁸ 21 gene-signature by Paik et al.¹⁹ (known as Oncotype DX) and 2 gene-signature by Ma et al.²⁰ While these signatures provide advances to our understanding of individual genes involved in resistance, they do not yet capture the complex interplay between biological mechanisms that governs tamoxifen resistance.

### SUMMARY

Prioritization of breast cancer patients based on the risk of resistance to tamoxifen plays a significant role in personalized therapeutic planning and improving disease course and outcomes. It is shown herein that a genome-wide pathway-centric computational framework identifies molecular pathways as markers of tamoxifen resistance in ER+ breast cancer patients. Through the association of pathway activity and response to tamoxifen, five biological pathways were identified and their ability to predict the risk of tamoxifen resistance in two independent patient cohorts (Test cohort1: log-rank p-value=0.02, adjusted HR=3.11; Test cohort2: log-rank p-value=0.01, adjusted HR=4.24) demonstrated. These pathways are not markers of aggressiveness and outperform known markers of tamoxifen response. The identified pathways and genes within these pathways can be utilized to prioritize patients who would benefit from tamoxifen treatment and patients at risk of tamoxifen resistance that should be provided alternative regimens.

In some examples, the methods include measuring expression of ER+ breast cancer-related molecules from ER+ breast cancer-related pathways in a sample obtained from a subject having ER+ breast cancer. ER+ breast cancer-related pathways can include retrograde neurotrophin signalling, loss of NLP from mitotic centrosomes, RNA polymerase III transcription initiation from Type 2 promoter, EIF2 pathway, and valine, leucine and isoleucine biosynthesis pathways. In some examples, the ER+ breast cancer-related molecules include adaptor-related protein complex 2, sigma-1 subunit (AP2S1) from the retrograde neurotrophin signaling pathway, cyclin-dependent kinase 2 (CDC2) from the loss of NLP from mitotic centrosomes pathway, general transcription factor IIIC subunit 3 (GTF3C3) from the RNA polymerase III transcription initiation from Type 2 promoter pathway, eukaryotic translation initiation factor 2-alpha kinase 3 (EIFA2AK3) from the EIF2 pathway, and leucyl-tRNA synthetase (LARS) from the valine, leucine and isoleucine biosynthesis pathway.

In some examples, measuring expression of ER+ breast cancer-related molecules from ER+ breast cancer-related pathways in a sample obtained from a subject having ER+ breast cancer further includes summing expression of AP2S1, expression of CDC2 multiplied by three, expression of GTF3C3, expression of EIFA2AK3, and expression of LARS multiplied by two to calculate a risk score for the ER+ breast cancer-related molecules for the sample obtained from a subject with ER+ breast cancer. The method can further include comparing the risk score to a value or threshold (such as a control value or control risk score) representing expression for the ER+ breast cancer-related molecules expected in a sample from a subject who will develop resistance to tamoxifen or control representing expression for the ER+ breast cancer-related molecules expected in a sample from a subject who will not develop resistance to tamoxifen. In some examples, a control risk score is calculated by summing expression of AP2S1, expression of CDC2 multiplied by three, expression of GTF3C3, expression of EIFA2AK3, and expression of LARS multiplied by two to calculate a risk score for the ER+ breast cancer-related molecules for samples obtained from subjects with ER+ breast cancer to form a risk score data set; calculating a mean of the risk score data set; calculating a standard deviation of the risk score data set; and summing the mean of the risk score data set with the standard deviation of the risk score data set to yield the control risk score, wherein the control representing expression for the ER+ breast cancer-related molecules expected in a sample from a subject who will develop resistance to tamoxifen is values greater than the control risk score, and wherein the control representing expression for the ER+ breast cancer-related molecules expected in a sample from a subject who will not develop resistance to tamoxifen is values less than or equal to the control risk score. In some examples, the control risk score is 4.5.

In some examples, such methods further include comparing expression of the ER+ breast cancer-related molecules from ER+ breast cancer-related pathways to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen. If expression of the ER+ breast cancer-related molecules is decreased relative to the control representing expression for the ER+ breast cancer-related molecules expected in a sample from a subject who will develop resistance to tamoxifen, or is similar to the control representing expression for the ER+ breast cancer-related molecules expected in a sample from a subject who will not develop resistance to tamoxifen, identifies the subject as one who will not develop resistance to tamoxifen. In some examples, such methods further include comparing expression of the ER+ breast cancer-related molecules from ER+ breast cancer-related pathways to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen. If expression of the ER+ breast cancer-related molecules is similar to the control representing expression for the ER+ breast cancer-related molecules expected in a sample from a subject who will develop resistance to tamoxifen, or is increased relative to the control representing expression for the ER+ breast cancer-related molecules expected in a sample from a subject who will not develop resistance to tamoxifen, the subject is identified as a subject with ER+ breast cancer who will develop resistance to tamoxifen.

The methods can include treating the subject with ER+ breast cancer. For example, the method can include administering a therapeutically effective amount of tamoxifen to the subject, thereby treating the subject with ER+ breast cancer, if it is determined that expression of the ER+ breast cancer-related molecules is decreased relative to a control representing expression of the ER+ breast cancer-related molecules expected in a sample from a ER+ breast cancer that develops resistance to the tamoxifen. In another example, the method can include administering a therapeutically effective amount of a non-tamoxifen therapy to the subject, thereby treating the subject with ER+ breast cancer, if it is determined that expression of the ER+ breast cancer-related molecules is increased relative to a control representing expression of the ER+ breast cancer-related molecules expected in a sample from a ER+ breast cancer that does not develop resistance to the tamoxifen. Exemplary non-tamoxifen therapies include alternative endocrine therapy, radiation therapy, and/or other chemotherapy. In some examples, the non-tamoxifen therapy includes a therapeutically effective amount of fulvestrant, a CDK 4/6 inhibitor, a PI3K inhibitor, luteinizing-hormone releasing hormone (LHRH) agonist, aromatase inhibitor (e.g., anastrozole or letrozole), radiation therapy or combinations thereof.

Any ER+ breast cancer (i.e., a breast cancer that has estrogen receptors) of any stage can be analyzed using the disclosed methods, such as one with low levels of Ki-67 (luminal A subtype), high levels of Ki-67 (luminal B subtype), progesterone receptor (PR) positive, PR negative, HER2 positive, HER2 negative, or combinations thereof. In one example, the ER+ breast cancer is triple-positive (ER-positive, PR-positive, and HER2-positive). In one example, the ER+ breast cancer is also PR-positive. In one example the ER+ breast cancer is a ductal carcinoma in situ (DCIS). In some examples, the ER+ breast cancer is an adenocarcinoma.

In some examples, a subject who will develop resistance to tamoxifen is one who will have a recurrence of their ER+ breast cancer within one year of treatment with the tamoxifen. In some examples, a subject who will not develop resistance to tamoxifen is one who will not have a recurrence of their ER+ breast cancer within one year of treatment with the tamoxifen.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color.

FIG. 1 shows a schematic representation of the utilization of independent patient cohorts for Training, Testing, and negative-control purposes utilized herein.

FIGS. 2A-2B. Comprehensive threshold analysis identifies pathway significance level. Threshold analysis in the Training cohort utilizing Cox proportional hazards model on the group of pathways (starting from the most significant pathways and adding the next most significant pathway, one at a time). Cutoff point was determined as a point on the graph when adding any additional pathway would not improve the model significance. Adjusted hazard p-value (A) and adjusted Cox Wald p-value (B) are used as threshold-deciding criteria.

FIGS. 3A-3B. Schematic representation of the pathway-centric approach. (a) Training phase: identification of molecular pathways of tamoxifen resistance. (b) Testing phase: clinical validation of identified candidate pathways and multi-modal prediction evaluation.

FIGS. 4A-4C. Training phase: pathway-centric approach identifies five biological pathways that govern tamoxifen response. (A) Schematic representation of the Testing phase of the disclosed approach: (left) patient molecular profiles are collected and analyzed; (middle) pathway activities are estimated in each patient using single-patient pathway enrichment analysis; (right) pathway activities are associated with response to tamoxifen using Cox proportional hazards modeling and are adjusted to common covariates, including age, tumor grade, tumor size (>2 cm vs ≤2 cm), lymph node status, and PR status. (B) Graphical illustration of tamoxifen-related treatment response or follow-up. Time to event (top): time interval between tamoxifen administration and earliest relapse is indicated by green line. Time to follow-up (bottom): time interval between tamoxifen administration and latest follow-up date is indicated by brown line (no tamoxifen-related events observed). (C) Heatmap representation of the pathway activity levels (i.e., NES) and their association with time to tamoxifen-related relapse or follow-up, in the Training cohort. Green line (left) marks the group of patients with tamoxifen-related relapse, sorted from the shortest to the longest time to relapse. Brown line (right) marks the group of patients with follow-up and without disease relapse until the latest follow-up, sorted from the shortest to longest time to follow-up.

FIG. 5 Graphical representation of the five pathways and their significantly contributing genes. Network-based representation of the five pathways. Selected genes shown (brown nodes) correspond to genes that contribute to significant enrichment of each pathway in the patient single-sample signatures. Node sizes represent number of times each gene appears in the leading edge in the single-sample pathway enrichment analysis (i.e., indicating significant changes in activity of this pathway across Training cohort). The list of genes in each pathway is listed below

| | |
|---|---|
| REACTOME RETROGRADE NEUROTROPHIN | DNM1 |
| SIGNALLING | SH3GL2 |
| | AP2M1 |
| | CLTA |
| | AP2A2 |
| | CLTC |
| | AP2B1 |
| | DNAL4 |
| | AP2S1 |
| | AP2A1 |
| | NTRK1 |
| | NGF |
| REACTOME LOSS OF NLP FROM MITOTIC | PCM1 |
| CENTROSOMES | SFI1 |
| | DYNC112 |
| | CEP164 |
| | TUBA4A |
| | CEP192 |
| | DCTN3 |
| | CEP76 |
| | OFD1 |
| | TUBG1 |
| | SSNA1 |
| | PRKACA |
| | NEDD1 |
| | FGFR1OP |
| | PAFAH1B1 |
| | CDC2 |
| | DYNLL1 |
| | CSNK1E |
| | CLASP1 |
| | CEP290 |
| | PCNT |
| | DCTN1 |
| | CEP57 |
| | YWHAE |
| | AKAP9 |
| | PRKAR2B |
| | CDK5RAP2 |
| | DYNC1H1 |
| | CETN2 |
| | SDCCAG8 |
| | ALMS1 |
| | DCTN2 |
| | CEP70 |
| | CEP152 |
| | CEP135 |
| | TUBA1A |
| | HSP90AA1 |
| | YWHAG |
| | PLK1 |
| | CEP78 |
| | CENPJ |
| | NEK2 |
| | NDE1 |
| | ODF2 |
| | CKAP5 |
| | TUBB |
| | MAPRE1 |
| | CEP63 |
| | CSNK1D |
| | PPP2R1A |
| | PLK4 |
| | CEP250 |
| | ACTR1A |
| | CEP72 |
| REACTOME RNA POLYMERASE III TRANSCRIPTION | GTF3C1 |
| INITIATION FROM TYPE 2 PROMOTER | POLR3H |
| | LZTS1 |
| | POLR3E |
| | POLR3B |
| | POLR2F |
| | POLR2H |
| | POLR2E |
| | GTF3C2 |
| | BRF1 |
| | GTF3C5 |
| | GTF3C4 |
| | POLR2K |
| | POLR2L |
| | BDP1 |
| | GTF3C3 |
| | POLR3D |
| | POLR3A |
| | TBP |
| | POLR3F |
| EIF2PATHWAY | EIF2S3 |
| | PPP1CA |
| | EIF2S2 |
| | GSK3B |
| | EIF5 |
| | EIF2AK3 |
| | EIF2AK4 |
| | EIF2S1 |
| | EIF2B5 |
| | EIF2AK1 |
| | EIF2AK2 |
| VALINE LEUCINE AND ISOLEUCINE BIOSYNTHESIS | BCAT2 |
| | VARS2 |
| | LARS2 |
| | PDHA1 |
| | VARS |
| | PDHA2 |
| | PDHB |
| | IARS2 |
| | BCAT1 |
| | LARS |
| | IARS |

FIGS. 6A-6F. The five pathways predict patients at risk of tamoxifen resistance in independent patient cohorts. (A, D) T-SNE and subsequent k-means clustering of Test cohort 1 (A) and Test cohort 2 (D) based on activity levels of the five pathways demonstrates patient separation into two groups: orange group (with overall increased activity levels of the five pathways) and turquoise group (with overall decreased activity levels of the five pathways). (B, E) Kaplan-Meier treatment-related survival analysis comparing two patient groups in Test cohort 1 (B) and in Test cohort 2 (e). Log-rank p-values and adjusted hazard ratios are indicated. (C, F) Leave-one-out cross-validation (LOOCV) correctly identified patients with poor response to tamoxifen (orange) and patients with favorable response to tamoxifen (turquoise) in Test cohort 1 (c) and Test cohort 2 (f). Accuracy values (%) are indicated.

FIGS. 7A-7B. ROC analysis demonstrated significant separation of patient groups based on activity levels of the five pathways. ROC analysis to show significance of the separation between patient groups in (A) FIGS. 6A and (B) FIG. 6D. Area under the curve (AUC) is reported.

FIGS. 8A-8C. Five pathways do not predict and are not affected by overall disease aggressiveness. (A) T-SNE and subsequent k-means clustering based on the activity levels of the five pathways in the negative control cohort. (B) Kaplan-Meier survival analysis on negative control cohort confirms that the five pathways do not predict disease aggressiveness. Log-rank p-value and hazard ratio are indicated. (C) Multivariable Cox proportional hazards model representing analysis for five pathways adjusted for various prognostic signatures in breast cancer, including Wang et al. (76 prognostic markers, with 57 present on U133 Plus 2.0) and van′t Veer et al. (70 prognostic markers, with 53 present on U133 Plus 2.0). Adjusted hazard p-values are reported.

FIGS. 9A-9D. Stratified analysis demonstrates that predictive ability of the five pathways is not dependent on the PR and Luminal A/B status. Patients in Test cohort 1 were stratified based on their progesterone PR status: Progesterone-positive (A, B) and Progesterone-negative (C,D). T-SNE with subsequent k-means clustering on PR+ (A) and PR− (C) patient subgroups. Kaplan-Meier survival analysis for PR+ (B) and PR− (D) patient groups. Log-rank p-values are indicated.

FIGS. 10A-10C. Predictive ability of the five pathways outperforms markers from other methods and signatures of tamoxifen response. (A, B) Comparison of the predictive ability of the five pathways (blue) to the candidate identified by other approaches, including Epsi et al. extreme-responder analysis (green), Zhong et al. SVM-based method (brown) and Yu et al. PRES random forest-based method (pink), through unadjusted (A) and adjusted for common covariates (B) Cox proportional hazards model. P-values for unadjusted and adjusted hazard ratios are indicated. (C) Multivariable Cox proportional hazards model representing analysis for the five pathways adjusted for different predictive signatures of tamoxifen response, including Men et al. (10 predictive markers, with 9 present on U133 Plus 2.0), Paik et al. (Oncotype DX, 21 predictive markers), and Ma et al. (2 predictive markers). Adjusted hazard p-values are indicated.

FIG. 11 Gene read-out analysis identifies five genes for clinical testing. Scattered plots to identify read-out genes for each pathway. Each dot is a gene and its relationship to (i) activity levels of the pathway it belongs to (Spearman correlation p-value, x-axis) and (ii) tamoxifen response (i.e., Cox proportional hazards p-value, y-axis). All five pathways are indicated. Size of nodes reflects Fisher's combined joint p-values (from x and y axes).

FIGS. 12A-12E Risk scores of tamoxifen resistance identity patients with significant difference in treatment-related survival. (A) Schematic representation of the risk score to fail tamoxifen (i.e., treatment failure score). Top: read-out genes for candidate molecular pathways are assigned; Middle: expression values for the read-out genes are multiplied by their corresponding weights; Bottom: the weighted expression values are then summed and utilized to assign low/intermediate or high risk of failing tamoxifen. (B-E) Validation studies using risk scores in Test cohort 1 (B,C) and Test cohort 2 (D,E). (B) Distribution of risk scores in Test cohort 1, with low/intermediate and high risk patients indicated. (C) Kaplan-Meier survival analysis, comparing low-intermediate and high risk patient groups in Test cohort 1. (D) Distribution of risk scores in Test cohort 2, with low/intermediate and high risk patients indicated. (E) Kaplan-Meier survival analysis, comparing low-intermediate and high risk patient groups in Test cohort 2.

### SEQUENCE LISTING

The nucleic sequences listed in the accompanying sequence listing are shown using standard letter abbreviations for nucleotide bases, as defined in 37 C.F.R. 1.822. Only one strand of each nucleic acid sequence is shown, but the complementary strand is understood as included by any reference to the displayed strand. The sequence listing submitted herewith generated on Jun. 15, 2021, 21 kb, is part of the disclosure and herein incorporated by reference.

SEQ ID NO: 1 is an exemplary AP2S1 sequence. GenBank® Accession No. BC006337.2.

SEQ ID NO: 2 is an exemplary CDC2 sequence. GenBank® Accession No. NM_001786.5

SEQ ID NO: 3 is an exemplary EIF2AK3 sequence. GenBank® Accession No. NM_004836.7

SEQ ID NO: 4 is an exemplary GTF3C3 sequence. GenBank® Accession No. NM_012086.5

SEQ ID NO: 5 is an exemplary LARS sequence. GenBank® Accession No. D84223.1

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Jun. 30, 2020. All references, including journal articles, patents, and patent publications, and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Adaptor-related protein complex 2, sigma-1 subunit (AP2S1): e.g., OMIM 602242. One of two major clathrin-associated adaptor complexes, AP-2, is a heterotetramer which is associated with the plasma membrane. This complex is composed of two large chains, a medium chain, and a small chain. AP2S1 encodes the small chain of this complex. AP2S1 plays a role in the retrograde neurotrophin signaling pathway. AP2S1 nucleic acids and proteins are included. Exemplary AP2S1 DNA, mRNA, and proteins include GENBANK® sequences NG_033136.1, BC006337.2, and P53680.2 respectively. There are several isoforms of AP2S1, including isoforms 3 (NM_001301076.2, NP_001288005.1), 4 (NM_001301078.2, NP 001288007.1), 5 (NM_001301081.2, NP_001288010.1), and AP17 (NM_004069.6, NP_004060.2). One of ordinary skill in the art can identify additional AP2S1 nucleic acid and protein sequences, including AP2S1 variants that retain biological activity (such as involvement in the retrograde neurotrophin signaling pathway). In some examples, AP2S1 is upregulated (e.g., expression of AP2S1 mRNA is increased) in an ER+ breast cancer that will develop resistance to tamoxifen chemotherapy, as compared to such expression in a ER+ breast cancer that will not develop resistance to tamoxifen chemotherapy.

Adenocarcinoma: Carcinoma derived from glandular tissue or in which the tumor cells form recognizable glandular structures. Adenocarcinomas can be classified, according to the predominant pattern of cell arrangement as papillary, alveolar, etc., or according to a particular product of the cells, as mucinous adenocarcinoma. Adenocarcinomas arise in several tissues, including the kidney, breast, colon, cervix, esophagus, gastric, pancreas, prostate, and lung.

Administration/delivery: To provide or give a subject an agent or therapy by any effective route. Examples of agents include chemotherapy, surgery, radiation therapy, targeted therapy, immunotherapy, or palliative care. Administration includes acute and chronic administration as well as local and systemic administration. In some examples, administration of a therapeutic agent, such as chemotherapy, is by injection (e.g., intravenous, intramuscular, intraosseous, intratumoral, or intraperitoneal). In some examples, administration therapeutic agent, such as chemotherapy, is oral, transdermal, or rectal. In some examples, administration therapeutic agent, such as tamoxifen, is oral.

Animal: Living multi-cellular vertebrate organisms, a category that includes, for example, mammals and birds. The term mammal includes both human and non-human mammals. Similarly, the term “subject” includes both human and veterinary subjects.

Breast Tumor: A neoplastic condition of breast tissue that can be benign or malignant. The most common type of breast cancer is breast carcinoma, such as ductal carcinoma. Ductal carcinoma in situ is a non-invasive neoplastic condition of the ducts. Lobular carcinoma is not an invasive disease but is an indicator that a carcinoma may develop. Infiltrating (malignant) carcinoma of the breast can be divided into stages (I, IIA, IIB, IIIA, IIIB, and IV). See, for example, Bonadonna et al., (eds), Textbook of Breast Cancer: A clinical Guide the Therapy, 3rd; London, Tayloy & Francis, 2006.

In some examples, a breast tumor is a breast cancer, such as a breast cancer that is positive for estrogen receptor (ER+). An ER+ breast cancer can be progesterone receptor positive (PR+) or negative (PR−). In some examples, an ER+ breast cancer is HER2−. In some examples, an ER+ breast cancer is HER2+. In some examples, an ER+ breast cancer is HER1−. In some examples, an ER+ breast cancer is HER1+.

Exemplary therapies for breast cancer include surgery (e.g., removal of some or all of the tumor), hormone blocking therapy (e.g., tamoxifen), radiation, cyclophosphamide plus doxorubicin (Adriamycin), taxane (e.g., docetaxel), and monoclonal antibodies such as trastuzumab (Herceptin) or pertuzumab, or combinations thereof. In some example, an ER+ breast cancer is treated with tamoxifen (such as tamoxifen citrate).

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, cancer cells, for example ER+ breast cancer cells, are analyzed by the disclosed methods.

Cell division cycle protein 2 homolog 2 (CDC2): e.g., OMIM 116940. Also known as CDK1 (cyclin dependent kinase 1). A highly conserved protein that functions as a serine/threonine kinase and is involved in cell cycle regulation, such as the loss of ninein-like protein (NLP) from mitotic centrosomes pathway. CDC2 nucleic acids and proteins are included. Exemplary CDC2 DNA, mRNA, and proteins include GENBANK® sequences AF512554.1, NM_001786.5 and AAM34793.1, respectively. There are several isoforms of CDC2, including isoforms 1-6 (e.g., NM_001786.5; NM_033379.4, NR_046402.2, NM_001170406.1, NM_001170407.1, NM_001320918.1). One of ordinary skill in the art can identify additional CDC2 nucleic acid and protein sequences, including CDC2 variants that retain biological activity (such as involvement in the loss of NLP from mitotic centrosomes pathway). In some examples, CDC2 is upregulated (e.g., expression of CDC2 mRNA is increased) in an ER+ breast cancer that will develop resistance to tamoxifen chemotherapy, as compared to such expression in a ER+ breast cancer that will not develop resistance to tamoxifen chemotherapy.

Control: A reference standard. In some embodiments, the control is a healthy subject. In other embodiments, the control is a subject with a ER+ breast cancer that is resistant to tamoxifen. In some embodiments, the control is a subject who responds positively to tamoxifen, such as a subject who does not develop resistance to tamoxifen. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference.

Detect: To determine if an agent (such as a signal; particular nucleotide; amino acid; nucleic acid molecule; mRNA; or protein) is present or absent. In some examples, detection can include further quantification. For example, use of the disclosed methods in particular examples permits detection of nucleic acid expression (e.g., mRNA expression) or protein expression in a sample, such as one from a subject with ER+ breast cancer.

Differential Expression: A nucleic acid molecule is differentially expressed when the amount of one or more of its expression products (e.g., transcript, such as mRNA, and/or protein) is higher or lower in one sample (such as a test ER+ breast cancer sample) as compared to another sample (such as a control ER+ breast cancer sample, such as one known to be resistant or not to tamoxifen). Detecting differential expression can include measuring a change in gene (such as by measuring mRNA) or protein expression.

Eukaryotic translation initiation factor 2-alpha kinase 3 (EIF2AK3): e.g., OMIM 604032. Also known as protein kinase R (PKR)-like endoplasmic reticulum kinase (PERK). EIF2AK3 phosphorylates the alpha subunit of eukaryotic translation-initiation factor 2 (EIF2), leading to its inactivation, and thus to a rapid reduction of translational initiation and repression of global protein synthesis. EIF2AK3 is a type I membrane protein located in the endoplasmic reticulum (ER), where it is induced by ER stress caused by malfolded proteins. EIF2AK3 nucleic acids and proteins are included. Exemplary EIF2AK3 DNA, mRNA, and proteins include GENBANK® sequences AH009678.2, NM_004836.7, and NP_004827.4, respectively. There are several isoforms of EIF2AK3, including isoforms 1-2 (e.g., NM 004836.7; NM_001313915.1). One of ordinary skill in the art can identify additional EIF2AK3 nucleic acid and protein sequences, including EIF2AK3variants that retain biological activity (such as regulation and phosphorylation of EIF2). In some examples, EIF2AK3 is upregulated (e.g., expression of EIF2AK3 mRNA is increased) in an ER+ breast cancer that will develop resistance to tamoxifen chemotherapy, as compared to such expression in a ER+ breast cancer that will not develop resistance to tamoxifen chemotherapy.

Expression: Translation of a nucleic acid into a peptide or protein. Peptides or proteins may be expressed and remain intracellular, become a component of the cell surface membrane, or be secreted into the extracellular matrix or medium.

General transcription factor IIIC subunit 3 (GTF3C3): e.g., OMIM 604888. The GTF3C3 gene encodes a subunit of the DNA-binding subcomplex (TFIIIC2) of transcription factor IIIC (TFIIIC). The TFIIIC complex mediates transcription of class III genes through direct recognition of promoters or promoter-TFIIIA complexes and subsequent recruitment of TFIIIB and RNA polymerase III. GTF3C3 plays a role in the RNA polymerase III transcription initiation from Type 2 promoter pathway. GTF3C3 nucleic acids and proteins are included. Exemplary GTF3C3 DNA, mRNA, and proteins include GENBANK® sequences NG_030373.1, NM_012086.5, and Q9Y5Q9.1, respectively. There are several isoforms of GTF3C3, including isoforms 1-2 (e.g., NM_012086.5; NM_001206774.2). One of ordinary skill in the art can identify additional GTF3C3 nucleic acid and protein sequences, including GTF3C3 variants that retain biological activity (such as involvement in the RNA polymerase III transcription initiation from Type 2 promoter pathway). In some examples, GTF3C3 is upregulated (e.g., expression of GTF3C3 mRNA is increased) in an ER+ breast cancer that will develop resistance to tamoxifen chemotherapy, as compared to such expression in a ER+ breast cancer that will not develop resistance to tamoxifen chemotherapy.

Leucyl-tRNA synthetase (LARS): e.g., OMIM 151350. The LARS gene encodes a cytoplasmic amino-acyl tRNA synthetase enzyme (aaRS) called LeuRS. LeuRS is one of several aaRS proteins that form a macromolecular multisynthetase complex that regulates transcription, translation, and various signaling pathways. LARS is involved in the synthesis of valine, leucine and isoleucine. LARS nucleic acids and proteins are included. Exemplary LARS DNA, mRNA, and proteins include GENBANK® sequences NG_042294.1 D84223.1 and BAA95667.1, respectively. There are several isoforms of LARS, including isoforms 1-4 (e.g., NM_020117.11; NM_016460.3, NM_001317964.1, NM_001317965.1). One of ordinary skill in the art can identify additional LARS nucleic acid and protein sequences, including LARS variants that retain biological activity (such as involvement in the synthesis of valine, leucine and isoleucine). In some examples, LARS is upregulated (e.g., expression of LARS mRNA is increased) in an ER+ breast cancer that will develop resistance to tamoxifen chemotherapy, as compared to such expression in a ER+ breast cancer that will not develop resistance to tamoxifen chemotherapy.

Primer: Short nucleic acids, for example DNA oligonucleotides 10 nucleotides or more in length, which are annealed to a complementary target nucleic acid strand (e.g., of a AP2S1, CDC2, GTF3C3, EIFA2AK3, or LARS nucleic acid molecule, such any of SEQ ID NOS: 1-5 or their complementary strand) by nucleic acid hybridization to form a hybrid between the primer and the target nucleic acid strand, then extended along the target nucleic acid strand by a polymerase enzyme. Therefore, primers can be used to measure nucleic acid expression. In addition, primer pairs can be used for amplification of a nucleic acid sequence, e.g., by the polymerase chain reaction (PCR) or other nucleic-acid amplification methods.

Primers include at least 10 nucleotides complementary to the target nucleic acid molecule. In order to enhance specificity, longer primers may also be employed, such as primers having 15, 20, 30, 40, 50, 60, 70, 80, 90 or 100 consecutive nucleotides of the complementary nucleic acid molecule to be detected. Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

In some examples, if the nucleic acid to be detected is DNA, the primer is DNA, RNA, or a mixture of both. In some examples, if the nucleic acid to be detected is RNA, the primer is RNA or DNA.

In some examples, primers include a detectable label, such as a fluorophore or enzyme, and are referred to as probes, which can also be used to detect a target nucleic acid molecule provided herein.

Sample or biological sample: A sample of biological material obtained from a subject, which can include cells, proteins, and/or nucleic acid molecules (such as DNA and/or RNA, such as mRNA). Biological samples include clinical samples useful for detection of disease, such as cancer, in subjects. Appropriate samples include any conventional biological samples, including clinical samples obtained from a human or veterinary subject. Exemplary samples include, without limitation, cancer samples (such as from surgery, tissue biopsy, tissue sections, or autopsy), cells, cell lysates, blood smears, cytocentrifuge preparations, cytology smears, bodily fluids (e.g., blood, plasma, serum, saliva, sputum, lymph node, urine, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as breast cancer). In a specific example, the sample is an ER+ breast cancer sample, such as a fine needle aspirate, core needle biopsy, stereotactic biopsy, surgical biopsy, or tissue sample.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166+1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15+20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG filtering (Wootton and Federhen, Meth. Enzymol. 266:554-571, 1996). In addition, a manual alignment can be performed.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 80%, at least 90%, at least 95%, at least 98%, or at least 99% sequence identity to a molecule listed in the sequence listing, such as SEQ ID NO: 1, 2, 3, 4 or 5. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In one example, the subject treated and/or analyzed with the disclosed methods has cancer, such as ER+ breast cancer. In some examples, the subject has an ER+ breast cancer that responds positively to tamoxifen, such as an ER+ breast cancer that does not develop resistance to tamoxifen. In some examples, the subject has an ER+ breast cancer that develops resistance to tamoxifen, such as a subject who has a cancer recurrence while taking tamoxifen or subsequent to tamoxifen administration. In some examples, the subject with ER+ breast cancer is one that has had surgery to remove the ER+ breast cancer, but has not yet received tamoxifen therapy.

Therapeutically effective amount: The amount of an active ingredient (such as a tamoxifen chemotherapeutic agent) that is sufficient to effect treatment when administered to a mammal in need of such treatment, such as treatment of an ER+ breast cancer. The therapeutically effective amount will vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's sensorimotor function. The treatment may be assessed by objective or subjective parameters; including the results of a physical examination, neurological examination, or psychiatric evaluations. For example, treatment of a cancer can include decreasing the size, volume, or weight of a cancer, decrease the number, size, volume, or weight of metastases, or combinations thereof, for example relative to an absence of the treatment.

### Overview

Provided herein is a pathway-centric computational framework to elucidate tamoxifen resistance. It is demonstrated that it outperforms known gene-based approaches. In some examples, the disclosed pathway-based approach has one or more of the following advantages (i) its ability to identify a tightly connected cooperative group of genes unified by the same function;²¹⁻²³ (ii) studying molecular pathways, rather than individual genes, produces more reliable read-out outputs as they are less susceptible to experimental noise;²⁴ (iii) pathway-level view enhances our understanding of the biological mechanisms related to disease and treatment response;²⁵⁻²⁸ and finally (iv) looking at alterations in biological pathways enhances the likelihood of identifying potential therapeutic targets to preclude or overcome resistance.

A systematic pathway-centric computational framework was used to elucidate molecular pathways as markers of tamoxifen resistance in ER+ breast cancer patients. Through the analysis of pathway activity in each ER+ patient and their association with response to tamoxifen (n=53), five biological pathways were identified as pathways essential for tamoxifen resistance: Retrograde Neurotrophin Signalling, Loss of NLP from Mitotic Centrosomes, RNA Polymerase III Transcription Initiation from Type 2 Promoter, EIF2 pathway, and Valine, Leucine and Isoleucine Biosynthesis. The identified five (5) pathways were shown to predict the risk of tamoxifen resistance in two independent patient cohorts²⁹ (Test cohort 1, n=66: log-rank p-value=0.02, accuracy in leave one out cross-validation (LOOCV)=85.8%; Test cohort 2, n=77: log-rank p-value=0.01, accuracy in LOOCV=82.5%) and their independence from known covariates, such as age, tumor grade, tumor size, lymph node status, and PR status, as the absence of PR in ER+ tumor can be an indicator of HER2 activation and an aggressive phenotype¹⁶ (Test cohort 1, adjusted hazard ratio=3.11; Test cohort 2, adjusted hazard ratio=4.24). Furthermore, stratified Kaplan-Meier survival analysis on the PR+ and PR− patients as well as patients with Ki-67 low and Ki-67 high status was performed, and it is shown herein that the five pathways can predict risk of resistance to tamoxifen in each PR and Ki-67 group. As a negative control, it is shown that the identified five pathways did not classify patients simply based on the disease aggressiveness (log-rank p-value=0.7, hazard ratio=1.246) and that in fact pathways associated with disease aggressiveness do not overlap with the five pathways. The disclosed method was compared to other computational techniques to tackle treatment response, including Epsi et al.²⁷ (which utilized extreme-responder analysis, using tails of the treatment response distribution to define a treatment response signature), Zhong et al.³⁰ (which used Support Vector Machine approach as a base), and Yu et al.³¹ (which uses random forest approach as a base) and demonstrated that the disclosed method outperforms these techniques in predicting risk of resistance to tamoxifen. The disclosed pathway signature was compared to other known signatures of tamoxifen response¹⁸⁻²⁰ and the superiority of this pathway-based approach (adjusted hazard ratio=3.11, hazard p-value=0.0278) is shown. Thus, the identified five pathways can be used to prioritize patients who would benefit from tamoxifen treatment as their first-line therapy, and to identify patients at risk of tamoxifen resistance who should be offered an alternative regimen plan.

Provided herein are methods of treating a subject with an ER+ breast cancer. Such methods can include measuring expression of ER+ breast cancer-related molecules from ER+ breast cancer-related pathways in a sample obtained from a subject with ER+ breast cancer, wherein the ER+ breast cancer-related pathways include retrograde neurotrophin signalling, loss of NLP from mitotic centrosomes, RNA polymerase III transcription initiation from Type 2 promoter, EIF2 pathway, and valine, leucine and isoleucine biosynthesis. If expression of the ER+ breast cancer-related molecules is decreased relative to a control representing expression of the ER+ breast cancer-related molecules expected in a sample from an ER+ breast cancer that develops resistance to the tamoxifen, the method further includes administering a therapeutically effective amount of tamoxifen to the subject, thereby treating the subject with ER+ breast cancer. If expression of the ER+ breast cancer-related molecules is increased relative to a control representing expression of the ER+ breast cancer-related molecules expected in a sample from an ER+ breast cancer that does not develop resistance to the tamoxifen. The method further includes administering a therapeutically effective amount of a non-tamoxifen therapy to the subject, thereby treating the subject with ER+ breast cancer. Exemplary non-tamoxifen therapies include one or more of a therapeutically effective amount of fulvestrant, a CDK 4/6 inhibitor, a PI3K inhibitor, luteinizing-hormone releasing hormone (LHRH) agonist, aromatase inhibitor, radiation therapy, or combinations thereof.

This provides personalized therapeutic advice for patients with ER+ breast cancer. Thus, subjects identified as having a high risk of developing resistance to tamoxifen can be treated with other therapies, for instance, alternative endocrine therapy, radiation therapy, or non-tamoxifen chemotherapy, while subject's with identified as having a low risk of developing resistance to tamoxifen can be treated with tamoxifen therapy.

Also provided are methods of identifying a subject with ER+ breast cancer who are or are not likely to develop resistance to tamoxifen. Such methods can include measuring expression of ER+ breast cancer-related molecules from ER+ breast cancer-related pathways in a sample obtained from a subject with ER+ breast cancer, wherein the ER+ breast cancer-related pathways include retrograde neurotrophin signalling, loss of NLP from mitotic centrosomes, RNA polymerase III transcription initiation from Type 2 promoter, EIF2 pathway, and valine, leucine and isoleucine biosynthesis. Expression of the ER+ breast cancer-related molecules from ER+ breast cancer-related pathways is determined. In some examples, the expression values are used to calculate a risk score, for example by weighting and summing the expression values. In some examples, a risk score for a sample analyzed using the disclosed methods is equal to the determined expression value for CDC2 multiplied by 3, the determined expression value obtained for LARS multiplied by 2, the determined expression value obtained for GTF3C3 used (e.g., multiplied by 1), the determined expression value obtained for AP2S1v, and the determined expression value obtained for EIF2AK3 used (e.g., multiplied by 1), and these five values summed. The resulting value is then used to determine if the subject is high risk, that is a subject with ER+ breast cancer who will likely develop resistance to tamoxifen, or low/intermediate risk, that is a subject with ER+ breast cancer who will not likely develop resistance to tamoxifen. In some examples, high risk and low risk values are determined, for example by obtaining gene expression data from a set of subjects with ER+ breast cancer and calculating risk scores for each sample in the gene expression data set. This set of risk scores forms a risk score data set. The mean and standard deviation can be calculated for the risk score data set. The mean+1 standard deviation of the risk score data set is calculated. If an individual subject's risk score is less than or equal to the mean+1 standard deviation of the risk score data set that subject is considered to be low/intermediate risk. If an individual subject's risk score is greater than to the mean+1 standard deviation of the risk score data set that subject is considered to be high risk.

In some examples, expression values of the ER+ breast cancer-related molecules from ER+ breast cancer-related pathways are compared to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen. If expression of the ER+ breast cancer-related molecules in the test subject is decreased relative to the control representing expression for the ER+ breast cancer-related molecules expected in a sample from a subject who will develop resistance to tamoxifen, or is similar to the control representing expression for the ER+ breast cancer-related molecules expected in a sample from a subject who will not develop resistance to tamoxifen, this identifies the test subject as who will not develop resistance to tamoxifen. In some examples the methods can further include administering tamoxifen to such subjects.

In some examples, expression of the ER+ breast cancer-related molecules from ER+ breast cancer-related pathways is compared to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen. If expression of the ER+ breast cancer-related molecules is similar to the control representing expression for the ER+ breast cancer-related molecules expected in a sample from a subject who will develop resistance to tamoxifen, or is increased relative to the control representing expression for the ER+ breast cancer-related molecules expected in a sample from a subject who will not develop resistance to tamoxifen, this identifies the test subject as who will likely develop resistance to tamoxifen. In some examples the methods can further include administering a non-tamoxifen therapy to such subjects.

In some examples, a subject likely to develop tamoxifen resistance is one who will likely have a treatment-related relapse free survival (tRFS) of less than one year, that is the interval between tamoxifen administration (e.g., immediately after surgery) and the earliest relapse (e.g., as local, regional, or distant metastasis) will be within one year. Thus, in some examples, a subject who will develop resistance to tamoxifen is one who has a recurrence of their ER+ breast cancer within one year of treatment with the tamoxifen. In some examples, a subject not likely to develop tamoxifen resistance is one who will likely have a treatment-related relapse free survival (tRFS) of more than one year (if at all), that is the interval between tamoxifen administration (e.g., immediately after surgery) and the earliest relapse, if any, (e.g., as local, regional, or distant metastasis) will be after one year. Thus, in some examples, a subject who will not develop resistance to tamoxifen is one who does not have a recurrence of their ER+ breast cancer within one year of treatment with the tamoxifen.

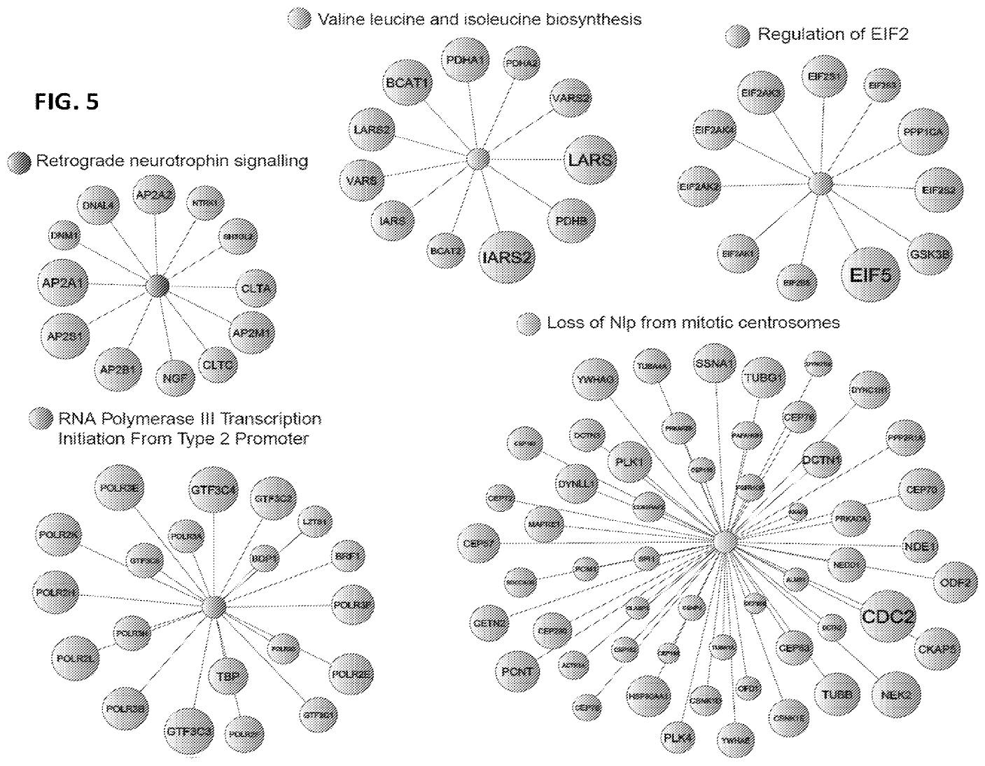

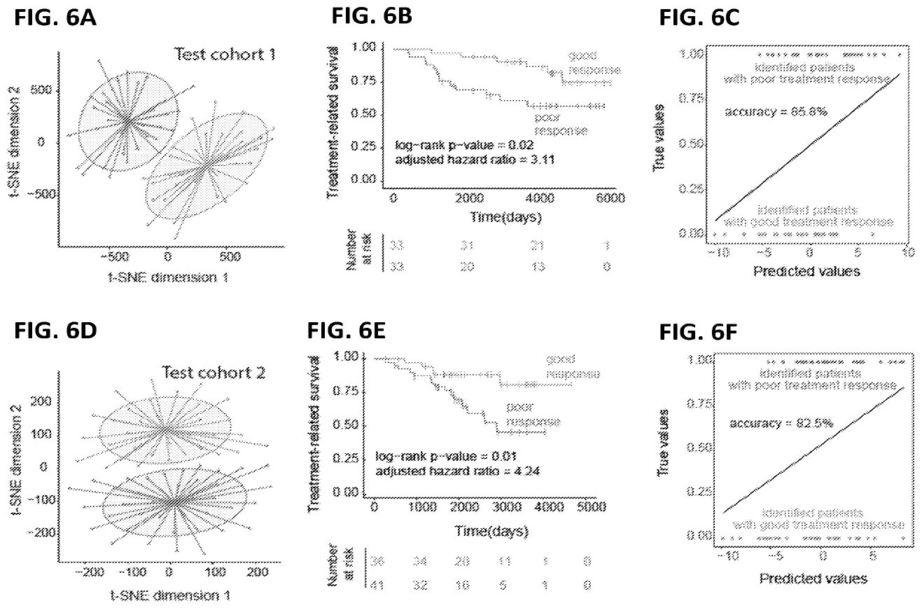

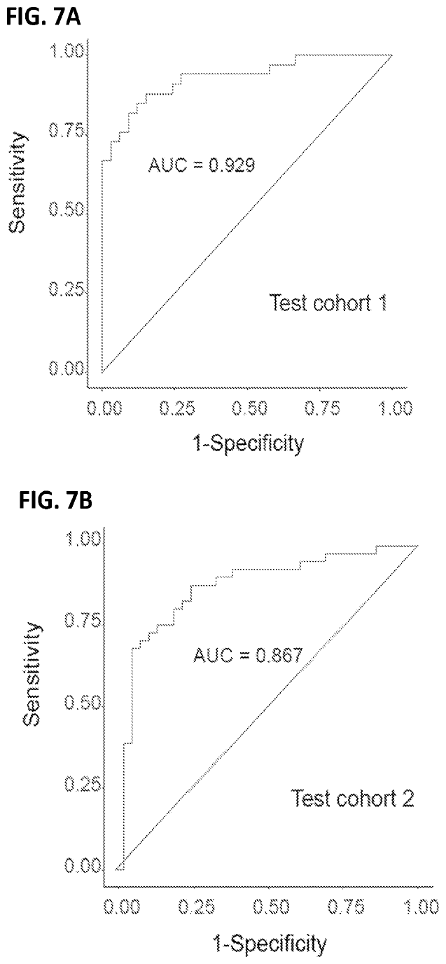

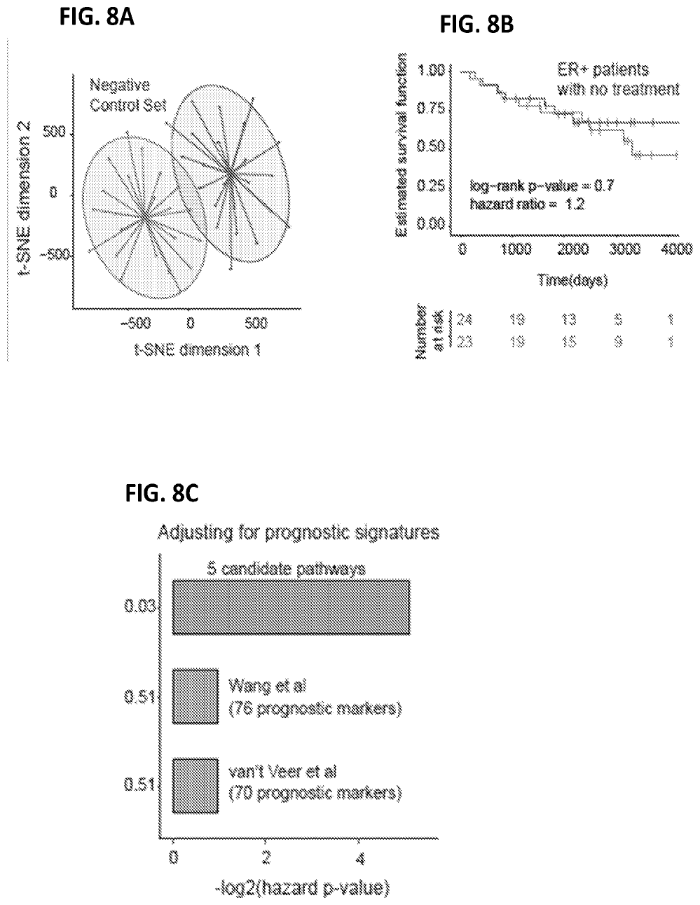

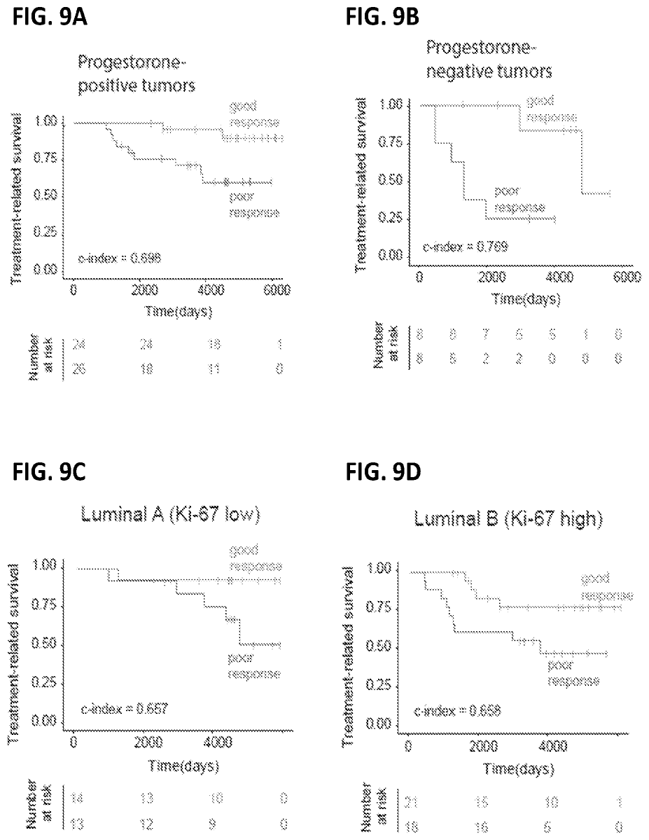

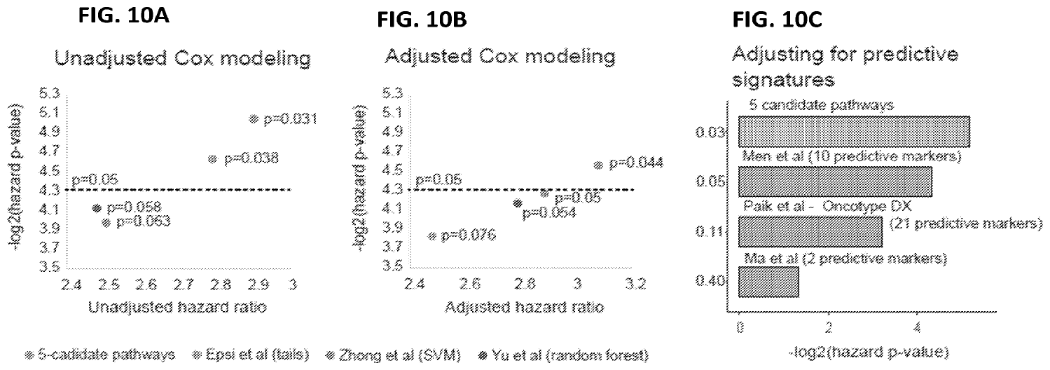

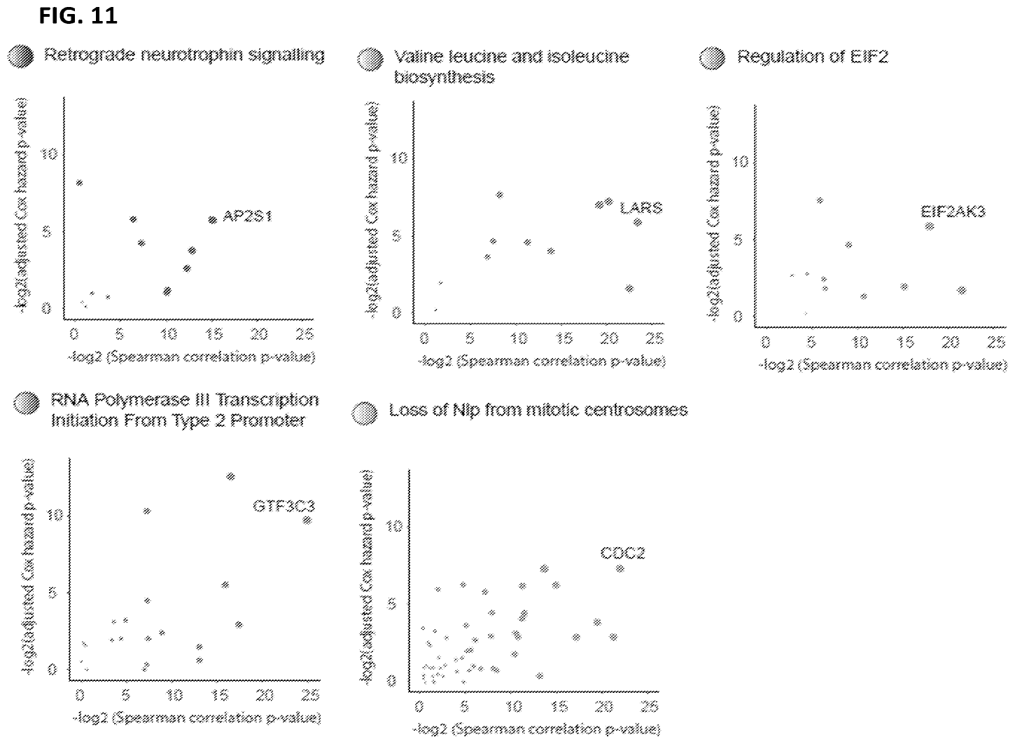

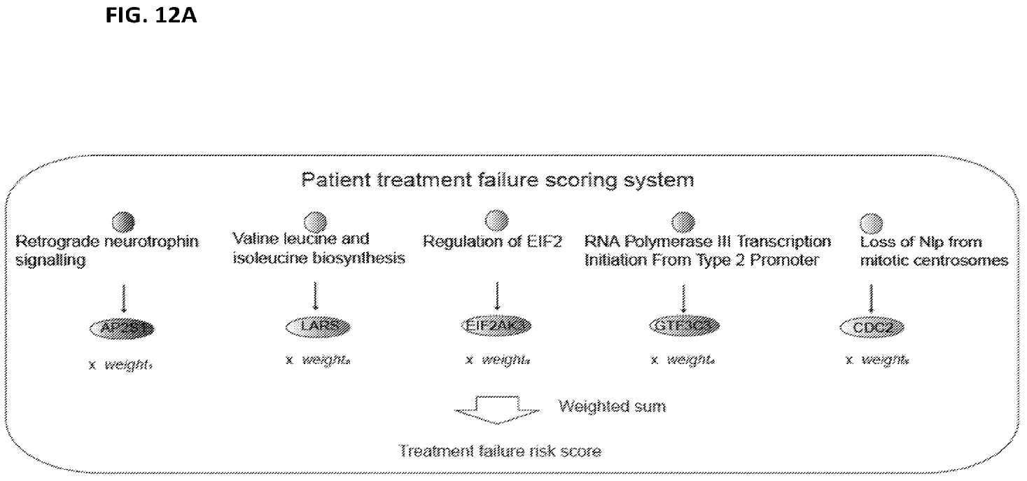

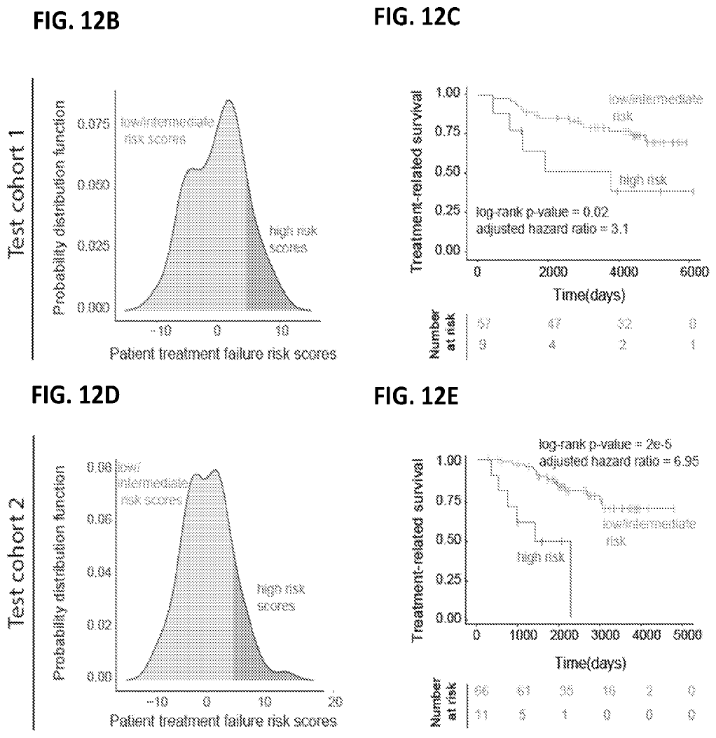

In some examples, the ER+ breast cancer-related pathways comprise adaptor-related protein complex 2, sigma-1 subunit (AP2S1) from the retrograde neurotrophin signaling pathway, cyclin-dependent kinase 2 (CDC2) from the loss of NLP from mitotic centrosomes pathway, general transcription factor IIIC subunit 3 (GTF3C3) from the RNA polymerase III transcription initiation from Type 2 promoter pathway, eukaryotic translation initiation factor 2-alpha kinase 3 (EIFA2AK3) from the EIF2 pathway, and leucyl-tRNA synthetase (LARS) from the valine, leucine and isoleucine biosynthesis pathway. Other examples are provided in FIG. 5

In some examples, the ER+ breast cancer analyzed using the disclosed methods has low levels of Ki-67 (luminal A subtype). In some examples, the ER+ breast cancer analyzed using the disclosed methods has high levels of Ki-67 (luminal B subtype). In some examples, the ER+ breast cancer analyzed using the disclosed methods is progesterone receptor (PR) positive. In some examples, the ER+ breast cancer analyzed using the disclosed methods is PR negative. In some examples, the ER+ breast cancer analyzed using the disclosed methods is triple positive. In some examples, the sample analyzed is an ER+ breast cancer sample or blood or lymph node sample.

In some examples, treating the subject only occurs where the subject is identified as a subject who will or will not develop tamoxifen resistance with a p value of at least 0.01 or at least 0.02.

Protein or nucleic acid expression can be analyzed, such as mRNA expression.

Evaluating Expression in a Subject with ER+ Breast Cancer

Provided herein are methods of identifying a subject with ER+ breast cancer who will (or will not) develop resistance to tamoxifen chemotherapy (such as a human or veterinary subject). In particular examples, the methods can determine with high accuracy whether a subject has an ER+ breast cancer that is likely (or unlikely) to develop resistance to tamoxifen. For example, the methods herein can distinguish between an ER+ breast cancer likely to develop resistance to tamoxifen from an ER+ breast cancer not likely to develop resistance to tamoxifen with an accuracy of at least 70%, at least 75%, at least 80%, at least 82%, or at least 85%. In one example, the methods herein between an ER+ breast cancer likely to develop resistance to tamoxifen from an ER+ breast cancer not likely to develop resistance to tamoxifen with an AOC curve of at least 0.8, at least 0.82, at least 0.85, at least 0.9 or at least 0.92. In one example, the methods herein between an ER+ breast cancer likely to develop resistance to tamoxifen from an ER+ breast cancer not likely to develop resistance to tamoxifen with a p value of less than 0.05, less than 0.04, less than 0.03, less than 0.02, or less than 0.01. The methods herein can be used to treat a variety of ER+ breast cancers with tamoxifen or non-tamoxifen therapy. It is helpful to determine whether or not an ER+ breast cancer in a subject will develop resistance to tamoxifen because there are a variety of protocols for treating ER+ breast cancer but if it is known that a patient will develop resistance to tamoxifen, alternative therapies can be administered. Hence, using the results of the disclosed methods allows subjects to be administered a therapy or treatment that will be effective for their ER+ breast cancer.

In some examples, an ER+ breast cancer that will not (or is likely to not) develop resistance to tamoxifen, is one that when treated with tamoxifen, does not have a detectable relapse of the ER+ breast cancer (e.g., local, regional, or distant metastasis) within 1 year, within 2 years, or within 5 years following surgery and subsequent administration of tamoxifen. In contrast, an ER+ breast cancer that will (or is likely to) develop resistance to tamoxifen, is one that when treated with tamoxifen, has a detectable relapse of the ER+ breast cancer (e.g., local, regional, or distant metastasis) within 1 year, within 2 years, or within 5 years following surgery and subsequent administration of tamoxifen.

In some examples, treatment of a subject with an ER+ breast cancer that will not, or is likely not to, develop resistance to tamoxifen, reduces the likelihood of recurrence of an ER+ breast cancer (such as a local or metastatic recurrence) within one year of tamoxifen treatment, such as by at least 20%, at least 50%, at least 80%, at least 90%, at least 95%, at least 98%, or even at least 100%, as compared to such as recurrence in a subject treated with tamoxifen who develops resistance to tamoxifen.

Examples of methods for treating a subject with ER+ breast cancer that will or will not develop tamoxifen resistance, or identifying a subject with ER+ breast cancer that will or will not develop tamoxifen resistance are disclosed herein are disclosed herein. In some examples, the subject with ER+ breast cancer is one that has had surgery to remove the ER+ breast cancer, but has not yet received tamoxifen therapy. In some examples, the methods include measuring expression of ER+ breast cancer-related molecules from ER+ breast cancer-related pathways in a sample obtained from a subject (such as an ER+ breast cancer sample). Expression of a variety of molecules from various pathways can be measured. Further, the methods can include measuring any number of molecules (e.g., a plurality of unique mRNAs or proteins). For example, expression of at least about two, at least about three, at least about four, at least about five, at least about six, at least about seven, at least about eight, at least about nine, at least about 10, at least about 15, at least about 20, at least about 25, at least about 50, at least about 100, at least about 200, at least about 500, or at least about 1000, or about 2-5, about 2 to 7, about 2-10, about 1-25, about 10-50, about 25-100, about 100-500, or about 100-1000, or about 3, 5, 6, or 7 different molecules can be measured. In some examples, expression of molecules from at least about two, at least about three, at least about four, at least about five, at least about six, at least about seven, at least about eight, at least about nine, at least about 10, at least about 15, at least about 20, at least about 25, at least about 50, at least about 100, at least about 200, at least about 500, or at least about 1000, or about 3-5, about 3-7, about 3-10, about 3-25, about 10-50, about 25-100, about 100-500, or about 100-1000, or about 3, 4, 5, 6, or 7 pathways can be measured. In one example, expression of molecules from 5 different ER+ breast cancer pathways are measured.

The methods herein can further include comparing the expression of ER+ breast cancer-related molecules (such as DNA, mRNA and proteins) from ER+ breast cancer-related pathways to such expression measured (or expected) in a control sample obtained from a subject (or population of subjects) with ER+ breast cancer known to develop or not develop resistance to tamoxifen, or to a historical control or reference value representing such expression expected in a subject (or population of subjects) with ER+ breast cancer know to develop or not develop resistance to tamoxifen. Thus, in some example, the methods include measuring expression of ER+ breast cancer-related molecules (such as DNA, mRNA and proteins) from ER+ breast cancer-related pathways in one or more control samples, such as a sample obtained from a subject (or population of subjects) with ER+ breast cancer known to develop resistance to tamoxifen and/or known to not develop resistance to tamoxifen.

Table 1 provides a summary of the ER+ breast cancer pathways, and specific genes whose expression can be analyzed to determine if the ER+ breast cancer will respond positively to the tamoxifen (i.e., not become resistant to tamoxifen). For example, mRNA expression and/or protein expression can be measured or detected in a ER+ breast cancer, and the expression compared to a control (e.g., representing expression observed in a ER+ breast cancer that develops resistance to tamoxifen) to determine if the ER+ breast cancer analyzed will develop resistance to tamoxifen or not (e.g., depending on whether there is an increase in expression as noted in the table).

TABLE 1
Exemplary ER+ breast cancer-related pathways and ER+ breast cancer-related
molecules with increased expression in an ER+ breast cancer that will develop resistance to
tamoxifen.

| | Cancer- | |
|---|---|---|
| Cancer-related | related | |
| pathways | molecules | mRNA Expression |
| Retrograde | AP2S1 | Increase by at least 25%, at least 30%, at least 40%, at |
| neurotrophin signalling | | least 50%, at least 55% (such as by 58.5%) in a breast |
| | | cancer that will develop tamoxifen resistance, as |
| | | compared to AP2S1 expression in a breast cancer that |
| | | will not develop tamoxifen resistance |
| Valine leucine and | LARS | Increase by at least at least 30%, at least 50%, at least |
| isoleucine biosynthesis | | 60%, at least 70%, at least 75% (such as by 79.6%) in a |
| | | breast cancer that will develop tamoxifen resistance, as |
| | | compared to LARS expression in a breast cancer that |
| | | will not develop tamoxifen resistance |
| Regulation of EIF2 | EIF2AK3 | Increase by at least 40%, at least 50%, at least 60%, at |
| | | least 70%, at least 75%, at least 80% (such as by |
| | | 80.1%) in a breast cancer that will develop tamoxifen |
| | | resistance, as compared to EIF2AK3 expression in a |
| | | breast cancer that will not develop tamoxifen resistance |
| RNA Polymerase II | GTF3C3 | Increase by at least at least 50%, at least 60%, at least |
| Transcrption Initiation | | 70%, at least 75%, at least 80%, at least 85%, at least |
| from Type 2 Promoter | | 90%, at least 95%, (such as by 95.7%) in a breast |
| | | cancer that will develop tamoxifen resistance, as |
| | | compared to GTF3C3 in a breast cancer that will not |
| | | develop tamoxifen resistance |
| Loss of Nlp from mitotic | CDC2/CDK1 | Increase by at least at least 40%, at least 50%, at least |
| centrosomes | | 60%, at least 70%, at least 75%, at least 80%, at least |
| | | 85% (such as by 86.2%) in a breast cancer that will |
| | | develop tamoxifen resistance, as compared to CDC2 in |
| | | a breast cancer that will not develop tamoxifen |
| | | resistance |

### Detecting Expression

As described herein, expression of any ER+ breast cancer-related molecule or combinations thereof disclosed herein (such as ER+ breast cancer-related molecules from ER+ breast cancer-related pathways that include (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as ER+ breast cancer-related molecules AP2S1, CDC2, GTF3C3, EIFA2AK3, and LARS) can be detected alone or in combination using a variety of methods. Expression of nucleic acid molecules (e.g., mRNA, cDNA) or protein is contemplated herein. Exemplary nucleic acid sequences that can be detected are provided in the sequence listing (e.g., SEQ ID NOS: 1-5). One skilled in the art can use these sequences to identify the corresponding mRNA and protein sequence encoded thereby, which can also be detected.

For example, expression of AP2S1 can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 1, for example by using probes or primers that can specifically hybridize to such sequences or the complementary strand thereof. Similarly, expression of AP2S1 can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 1, for example by using antibodies or fragments thereof that can specifically bind to such a protein. Expression of CDC2 can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 2, for example by using probes or primers that can specifically hybridize to such sequences or the complementary strand thereof. Similarly, expression of CDC2 can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 2, for example by using antibodies or fragments thereof that can specifically bind to such a protein. Expression of EIFA2AK3 can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 3, for example by using probes or primers that can specifically hybridize to such sequences or the complementary strand thereof. Similarly, expression of EIFA2AK3 can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 3, for example by using antibodies or fragments thereof that can specifically bind to such a protein. Expression of GTF3C3 can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 4, for example by using probes or primers that can specifically hybridize to such sequences or the complementary strand thereof. Similarly, expression of GTF3C3 can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 4, for example by using antibodies or fragments thereof that can specifically bind to such a protein. Expression of LARS can be determined by measuring expression of a nucleic acid molecule comprising at least 80%, at least 85%, at least 90%, at least 95%, at least 96%, at least 97%, at least 98%, at least 99%, or 100% sequence identity to SEQ ID NO: 5, for example by using probes or primers that can specifically hybridize to such sequences or the complementary strand thereof. Similarly, expression of LARS can be determined by measuring expression of a protein encoded by a sequence comprising at least 80%, at least 85%, at least 90%, at least 95%, at least 96%, at least 97%, at least 98%, at least 99%, or 100% sequence identity to SEQ ID NO: 5, for example by using antibodies or fragments thereof that can specifically bind to such a protein.

1. Methods for Detecting mRNA Expression

Gene expression can be evaluated by detecting mRNA encoding the gene of interest. Thus, the disclosed methods can include evaluating mRNA encoding ER+ breast cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS from these pathways. In some examples, mRNA expression is quantified.

RNA can be isolated from a cancer sample (such as an ER+ breast cancer) or other sample (e.g., blood or lymph node sample from a subject with ER+ breast cancer) from a subject, for example using commercially available kits, such as those from QIAGEN®. General methods for mRNA extraction are disclosed in, for example, including Ausubel et al., Current Protocols of Molecular Biology, John Wiley and Sons (1997). RNA can be extracted from paraffin embedded tissues (e.g., see Rupp and Locker, Lab Invest. 56:A67 (1987), and De Andres et al., BioTechniques 18:42044 (1995)). Total RNA from cells in culture (such as those obtained from a subject) can be isolated using QIAGIN® RNeasy mini-columns. Other commercially available RNA isolation kits include MASTERPURE®. Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from an ER+ breast cancer or other biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

In one example, RT-PCR can be used. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. In one example the internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, tubulin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

The steps of a representative protocol for quantifying gene expression using fixed, paraffin-embedded tissues as the RNA source, including mRNA isolation, purification, primer extension and amplification are given in various publications (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a tumor sample or other tissue sample. After analysis of the RNA concentration, RNA repair and/or amplification steps can be included, if necessary, and RNA is reverse transcribed using gene specific promoters followed by RT-PCR. The primers used for the amplification are selected so as to amplify a unique segment of the gene of interest, such as mRNA encoding cancer-related molecules from ER+ breast-cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. In some embodiments, expression of other genes is also detected. Primers that can be used to amplify ER+ cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis, such as AP2S1, CDC2, GTF3C3, EIFA2AK3, and/or LARS molecules are commercially available or can be designed and synthesized (such as based on SEQ ID NOS: 1-5). In some examples, the primers specifically hybridize to a promoter or promoter region of an ER+ breast-cancer-related molecule from an ER+ breast-cancer-related pathway, such as (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis, such as AP2S1, CDC2, GTF3C3, EIFA2AK3, and LARS.

Another quantitative nucleic acid amplification procedure is described in U.S. Pat. No. 5,219,727. Here, the amount of a target sequence in a sample (e.g., AP2S1, CDC2, GTF3C3, EIFA2AK3, and LARS) is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of one or more “housekeeping” genes or “internal controls” can also be evaluated. These terms include any constitutively or globally expressed gene whose presence enables an assessment of expression levels. Such an assessment includes a determination of the overall constitutive level of gene transcription and a control for variations in, for example RNA recovery. Exemplary housekeeping genes include beta-actin and tubulin.

In some examples, gene expression is identified or confirmed using a microarray technique. Thus, the expression profile can be measured in a biological sample, using microarray technology. In this method, nucleic acid sequences (including cDNAs and mRNAs) encoding ER+ breast-cancer-related molecules from ER+ breast-cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS are plated, or arrayed, on a microchip substrate. The arrayed sequences are then hybridized with specific nucleic acid probes from cells or tissues of interest. As in the RT-PCR method, an exemplary source of mRNA can be total RNA isolated from human ER+ breast cancers, and optionally from corresponding noncancerous tissue and normal tissues or cell lines.

In a specific embodiment of the microarray technique, PCR amplified inserts of cDNA clones are applied to a substrate in a dense array. At least probes specific for nucleotide sequences encoding ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS (and, in some examples, one or more housekeeping genes) are applied to the substrate, and the array can consist essentially of, or consist of these sequences. The microarrayed nucleic acids are suitable for hybridization under stringent conditions. Fluorescently labeled cDNA probes (e.g., specific for SEQ ID NOS: 1-5) may be generated through incorporation of fluorescent nucleotides by reverse transcription of RNA extracted from ER+ breast cancer tissues. Labeled cDNA probes applied to the chip hybridize with specificity to each spot of DNA on the array. After stringent washing to remove non-specifically bound probes, the chip is scanned by confocal laser microscopy or by another detection method. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for cancer-related molecules from ER+ breast cancer cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as P2S1, CDC2, GTF3C3, EIFA2AK3, and/or LARS. Microarray analysis can be performed by commercially available equipment, following manufacturer's protocols.

Serial analysis of gene expression (SAGE) allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997, herein incorporated by reference in their entireties).

In situ hybridization (ISH) is another method for detecting and comparing expression of ER+ breast cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as ER+ breast cancer tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS.

Sample cells or tissues can be treated to increase their permeability to allow one or more probes to enter the cells, such as gene-specific probes for ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. The probes are added to the treated cells, allowed to hybridize to target nucleic acid molecules in the samples at pertinent temperature, and excess probes washed away. The probes can be labeled, for example with a radioactive, fluorescent or antigenic tag, so that the probe's location and quantity in the tissue can be determined, for example using autoradiography, fluorescence microscopy or immunoassay. Probes can be designed such that the probes specifically bind a gene of interest because cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS are known. In some examples, probes that can specifically hybridize to SEQ ID NOS: 1-5 are used.

In situ PCR is the PCR-based amplification of the target ER+ breast cancer-related nucleic acid sequences prior to ISH. For detection of RNA, an intracellular reverse transcription step can be introduced to generate complementary DNA from RNA templates prior to in situ PCR. This enables detection of low copy RNA sequences.

Prior to in situ PCR, cells or tissue samples can be fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products can be achieved by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Gene expression can also be detected and quantitated using the nCounter® technology developed by NanoString (Seattle, Wash.; see, for example, U.S. Pat. Nos. 7,473,767; 7,919,237; and 9,371,563, which are herein incorporated by reference in their entireties). The nCounter® analysis system utilizes a digital color-coded barcode technology that is based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest (such as a TACE-response gene). Mixed together with controls, they form a multiplexed CodeSet.

Each color-coded barcode represents a single target molecule. Barcodes hybridize directly to target molecules and can be individually counted without the need for amplification. The method includes three steps: (1) hybridization; (2) purification and immobilization; and (3) counting. The technology employs two approximately 50 base probes per mRNA that hybridize in solution. The reporter probe carries the signal; the capture probe allows the complex to be immobilized for data collection. After hybridization, the excess probes are removed and the probe/target complexes are aligned and immobilized in the nCounter® cartridge. Sample cartridges are placed in the digital analyzer for data collection. Color codes on the surface of the cartridge are counted and tabulated for each target molecule. This method is described in, for example, U.S. Pat. No. 7,919,237; and U.S. Patent Application Publication Nos. 20100015607; 20100112710; 20130017971, which are herein incorporated by reference in their entireties. Information on this technology can also be found on the company's website (nanostring.com).

2. Arrays for Profiling Gene Expression

In particular embodiments, arrays (such as a solid support) are used to evaluate gene expression, for example to determine if a patient with ER+ breast cancer will develop resistance to tamoxifen (or not). Such arrays can include a set of specific binding agents (such as nucleic acid probes and/or primers specific for ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. When describing an array that consists essentially of probes or primers specific for ER+ breast cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS, such an array includes probes or primers specific for the ER+ breast cancer specific gene or genes (such as specific for each of AP2S1, CDC2, GTF3C3, EIFA2AK3, and LARS), and can further include control probes or primers, such as 1-10 control probes or primers (for example to confirm the reaction conditions are sufficient). In some examples, the array furthers includes at least 1, 2, 3, 4 or 5 additional probes for other genes, such as other genes associated with breast cancer (e.g., ER, PR, BRCA1, BRCA2, and/or Ki67). In some examples, the array includes 1-10 housekeeping-specific probes or primers. In one example, an array is a multi-well plate (e.g., 98 or 364 well plate).

In one example, the array includes, consists essentially of, or consists of probes or primers (such as an oligonucleotide or antibody) that can recognize ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS (and, in some examples, also 1-10 housekeeping genes). The oligonucleotide probes or primers can further include one or more detectable labels, to permit detection of hybridization signals between the probe and target sequence (such as cancer-related molecules from cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS.

a. Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567).

In one example, the solid support surface is polypropylene. In another example, a surface activated organic polymer is used as the solid support surface. One example of a surface activated organic polymer is a polypropylene material aminated via radio frequency plasma discharge. Such materials are easily utilized for the attachment of nucleotide molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

b. Array Formats

A wide variety of array formats can be employed. One example includes a linear array of oligonucleotide bands, generally referred to in the art as a dipstick. Another suitable format includes a two-dimensional pattern of discrete cells (such as 4096 squares in a 64 by 64 array). Other array formats including, but not limited to slot (rectangular) and circular arrays are equally suitable for use. In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit a low background fluorescence.

The array formats can be included in a variety of different types of formats. A “format” includes any format to which probes, primers or antibodies can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides.

The arrays of can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are describe in Matson et al., Anal. Biochem. 217:306-10, 1994. In one example, the oligonucleotides are synthesized onto the support using chemical techniques for preparing oligonucleotides on solid supports (such as see PCT applications WO 85/01051 and WO 89/10977, or U.S. Pat. No. 5,554,501).

The oligonucleotides can be bound to the polypropylene support by either the 3′ end of the oligonucleotide or by the 5′ end of the oligonucleotide. In one example, the oligonucleotides are bound to the solid support by the 3′ end. In general, the internal complementarity of an oligonucleotide probe in the region of the 3′ end and the 5′ end determines binding to the support.

In particular examples, the oligonucleotide probes on the array include one or more labels, that permit detection of oligonucleotide probe:target sequence hybridization complexes.

3. Detecting Protein Expression

In some examples, expression of ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS proteins is analyzed. Suitable biological samples include samples containing protein obtained from a ER+ breast cancer or other sample (e.g., blood or lymph node tissue) of a subject. An alteration in the amount of ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and/or LARS proteins in a ER+ breast cancer from the subject (and in some examples relative to a control, such as an increase or decrease in protein expression), indicates whether the ER+ breast cancer will develop resistance to tamoxifen (or not), as described herein (for example whether an expression score is above or below a threshold risk value).

Antibodies specific for cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS proteins can be used for protein detection and quantification, for example using an immunoassay method, such as those presented in Harlow and Lane (Antibodies, A Laboratory Manual, CSHL, New York, 1988). In some examples, antibodies are used that specifically bind to a protein encoded by any of SEQ ID NOS: 1-5.

Exemplary immunoassay formats include ELISA, Western blot, and RIA assays. Thus, protein levels of ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS proteins in a ER+ breast cancer sample can be evaluated using these methods. Immunohistochemical techniques can also be utilized protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

To quantify proteins, a biological sample of a subject that includes cellular proteins can be used. Quantification of ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS proteins can be achieved by immunoassay methods. The amount ER+ breast cancer-related protein from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS protein can be assessed in a ER+ breast cancer sample and optionally in ER+ breast cancer samples from patients known to develop or not develop resistance to tamoxifen. The amounts of ER+ breast cancer-related protein from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS protein in the ER+ breast cancer can be analyzed, for example compared to levels of the protein found in ER+ breast cancer samples from patients known to develop or not develop resistance to tamoxifen or other control (such as a standard value or reference value), such as a risk score threshold. A significant increase or decrease in the amount can be evaluated using statistical methods.

Quantitative spectroscopic approaches, such as SELDI, can be used to analyze expression of ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS expression in an ER+ breast cancer sample. In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

The surface chemistry allows the bound analytes to be retained and unbound materials to be washed away. Subsequently, analytes bound to the surface (such as tumor-associated proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind ER+ breast cancer-related proteins from ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. In other examples, the chromatographic surface consists essentially of, or consists of, antibodies that specifically bind ER+ breast cancer-related proteins from c ER+ breast cancer-related pathways, including (i) retrograde neurotrophin signalling, (ii) loss of NLP from mitotic centrosomes, (iii) RNA polymerase III transcription initiation from type 2 promoter, (iv) EIF2 pathway, and (v) valine, leucine and isoleucine biosynthesis pathways, such as AP2S1, CDC2, GTF3C3, EIFA2AK3, and LARS. In some examples, the chromatographic surface includes antibodies that bind other molecules, such as housekeeping proteins (e.g., tubulin, b-actin).

In another example, antibodies are immobilized onto the surface using a bacterial Fc binding support. The chromatographic surface is incubated with a sample, such as a sample of a lung or colon tumor. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

### B. Exemplary Samples

The methods provided herein include detecting expression (e.g., mRNA expression) of ER+ breast cancer-related molecules from ER+ breast cancer-related pathways, including (i) retrograde neurotrophin signalling, (ii) loss of NLP from mitotic centrosomes, (iii) RNA polymerase III transcription initiation from type 2 promoter, (iv) eukaryotic initiation factor 2 (EIF2) pathway, and (v) valine, leucine and isoleucine biosynthesis pathways, such as AP2S1, CDC2, GTF3C3, EIFA2AK3, and/or LARS ER+ breast cancer-related molecules in samples (such as ER+ breast cancer samples or other biological samples from an ER+ breast cancer subject, such as plasma, serum, or lymph node samples).

In some embodiments, the cancer samples (such as ER+ breast cancer samples) are obtained from subjects diagnosed with cancer (such as lung or colon cancer). A “sample” refers to part of a tissue that is either the entire tissue, or a diseased or healthy portion of the tissue. As described herein, cancer samples (such as ER+ breast cancer samples) can be used to calculate a risk score, compared to a control, or both. In some embodiments, the control is a ER+ breast cancer sample obtained from a subject or group of subjects known to have not developed tamoxifen resistance, or to have developed tamoxifen resistance.

In other embodiments, the control is a standard or reference value based on an average of historical values. In some examples, the reference values are an average expression (e.g., mRNA expression) value for each of an ER+ breast cancer-related molecule from ER+ breast cancer-related pathways, including (i) retrograde neurotrophin signalling, (ii) loss of NLP from mitotic centrosomes, (iii) RNA polymerase III transcription initiation from type 2 promoter, (iv) eukaryotic initiation factor 2 (EIF2) pathway, and (v) valine, leucine and isoleucine biosynthesis pathways, such as AP2S1, CDC2, GTF3C3, EIFA2AK3, and/or LARS in a sample (such as an ER+ breast cancer sample) obtained from a subject or group of subjects known to have not developed tamoxifen resistance, or to have developed tamoxifen resistance.

Tissue samples can be obtained from a subject, for example, from cancer patients (such as ER+ breast cancer patients) who have undergone tumor resection as a form of treatment. In some embodiments, cancer samples (such as ER+ breast cancer samples) are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, proteins and/or nucleic acid molecules (e.g., DNA, RNA, mRNA, and cDNA) are isolated or purified from the cancer sample (such as an ER+ breast cancer sample). In some examples, the cancer sample (such as an ER+ breast cancer sample) is used directly, or is concentrated, filtered, or diluted.

### 3. Treatment

In the methods herein, subjects identified as having an ER+ breast cancer that is not likely to develop resistance to tamoxifen, can be further treated with tamoxifen (e.g.., tamoxifen citrate). In some examples, tamoxifen is administered orally with or without food, such as once or twice daily for at least 2 years, at least 5 years, or at least 10 years. In some examples, a therapeutically effective amount is at least 5 mg per day, such as at least 10 mg, at least 20 mg, at least 30 mg or at least 40 mg/day, such as 5 to 40 mg or 20 to 40 mg per day. In some examples, a therapeutically effective amount is 20 per day for 2 years, 5 years, or 10 years. Daily dosages greater than 20 mg are usually divided in half and taken twice a day, in the morning and evening.

In the methods herein, subjects identified as having an ER+ breast cancer that is likely to develop resistance to tamoxifen, can be further treated with a non-tamoxifen therapy. That is, the subject is not administered tamoxifen. In some examples, such subjects are administered one or more of a selective estrogen receptor degrader (e.g., fluvestraint, for example via injection, such as monthly), a luteinizing-hormone releasing hormone (LHRH) agonist (e.g., goserelin or leuprolide), an aromatase inhibitor (e.g., letrozole, anastrozole, or exemestane, for example for at least 2 years, at least 5 years, or at least 10 years). In some examples fluvestraint is used in combination with a CDK 4/6 inhibitor (e.g., palbociclib, ribociclib, or abemaciclib) or PI3K inhibitor (e.g., alpelisib, for example orally), or radiation therapy.

### 4. Calculation of Risk Score

The methods herein can also be used to calculate patient risk scores. In some embodiments risk scores are based on read-out genes defined as those (i) whose expression levels significantly correlated with pathway activity changes, for corresponding pathways; (ii) that were significantly associated with response to tamoxifen. Read-out genes might be used to define a risk to develop resistance to tamoxifen. One method of calculating a risk score is to calculate the weighted sum of the read-out gene expression values.

$$risk score = {\sum }_{k = 1}^{\# of read out genes} x ( k ) * w ( k ) \qquad ( Equation 1 )$$

where k is a read-out gene, x(k) is the expression value of k, and w(k) is a weight. A gene expression value could be a normalized signal intensity value generated using Robust Microarray Analysis. Gene expression values can be measured in a variety of other ways, which are known to a person having skill in the art.

In some embodiments weights are defined based on the ability of the activity levels of candidate molecular pathways to efficiently distinguish patient clusters. A receiver operating characteristics (ROC) analysis is one method that can be used to define weights. The analysis might be performed using a multiple (i.e., multivariable) logistic regression model, where normalized enrichment scores (i.e., NESs) of the final candidate pathways are used as input parameters (i.e., independent/predictor variables) and patient clusters are utilized as a dependent/response variable. ROC curves might be evaluated using area under the curve (AUC) (Hanley et al., Radiology (1982) 143:29-36), where AUC score of 0.5 indicates a random predictor. A logistic regression analysis can be conducted using glm (Zeileis et al., J. Statistical Software (2008) 27:1-25) function, and ROC analysis might be performed using pROC (Robin, et al., BMC Bioinf. (2011) 12:77) and ggplot2 packages in R.

AUC values could be used to define the weights with higher AUC values corresponding to higher weight values. In some embodiments the weights are only whole numbers. In one such embodiment, AUC values of ≤0.749 are assigned a weight of 1, 0.75-0.799 assigned a weight of 2, 0.80-0.849 assigned a weight of 3, 0.85-0.899 assigned a weight of 4, and so on. In another embodiment weights are chosen based on the range of AUC values calculated.

In some examples, a risk score for a sample analyzed using the disclosed methods is equal to the determined expression value for CDC2 multiplied by 3, the determined expression value obtained for LARS multiplied by 2, the determined expression value obtained for GTF3C3 used (e.g., multiplied by 1), the determined expression value obtained for AP2S1v, and the determined expression value obtained for EIF2AK3 used (e.g., multiplied by 1), and these five values summed. The resulting value is then used to determine if the subject is high risk, that is a subject with ER+ breast cancer who will likely develop resistance to tamoxifen, or low/intermediate risk, that is a subject with ER+ breast cancer who will not likely develop resistance to tamoxifen. High risk and low risk values are determined, for example by obtaining gene expression data from a set of subjects with ER+ breast cancer and calculating risk scores for each sample in the gene expression data set. This set of risk scores forms a risk score data set. The mean and standard deviation can be calculated for the risk score data set. The mean+1 standard deviation of the risk score data set is calculated. If an individual subject's risk score is less than or equal to the mean+1 standard deviation of the risk score data set that subject is considered to be low/intermediate risk. If an individual subject's risk score is greater than to the mean+1 standard deviation of the risk score data set that subject is considered to be high risk.

In some embodiments the gene expression data set from a set of subjects with ER+ breast cancer is obtained from a publicly available GEO data repository (Barrett et al., Nucleic Acids Res. (2013) 41:D991-5) from multi-institutional multi-PI comprehensive Loi et al. study (Loi et al., J. Clin. Oncol. (2007) 25:1239) GSE6532: such as GUYT GSE6532 or OXFT-GSE6532. In another embodiment gene expression data is collected from a set of subjects with ER+ breast cancer and assembled into a new gene expression data set. In a further embodiment the mean+1 standard deviation is equal to 4.5 score.

### EXAMPLES

In the examples below, a pathway-centric genome-side computational approach uncovered biological pathways highly associated with risk of tamoxifen resistance in ER+ breast cancer patients. This approach identified a tightly connected group of genes, ER+ breast cancer biological pathways (and individual genes within each pathway), thus (i) decreasing the chances of experimental noise present in biological experiments; (ii) improving understanding of the mechanisms implicated in tamoxifen resistance in ER+ breast cancer; and (iii) increasing the likelihood of identifying a functionally relevant signature, which can be utilized to study mechanisms of primary resistance and their therapeutic targeting. Furthermore, these biological pathways are highly associated with a wide spectrum of treatment responses (as opposed to selecting a limited category of patients for analysis), reflecting heterogeneity of response to tamoxifen present in a clinical setting.

The computational analysis identified five molecular pathways implicated in tamoxifen resistance in ER+ breast cancer, including (i) retrograde neurotrophin signalling, (ii) loss of NLP from mitotic centrosomes, (iii) RNA polymerase III transcription initiation from type 2 promoter, (iv) eukaryotic initiation factor 2 (EIF2) pathway, and (v) valine, leucine and isoleucine biosynthesis. The retrograde neurotrophin signalling pathway is implicated in metabolic detoxification, mitosis, clathrin-mediated vesicles development.⁵² Ninein-like protein (NLP) (also known as NINL) is a part of the loss of NLP from mitotic centrosomes. The deregulated expression of NLP in cell models leads to mitotic spindle aberrations, spindle checkpoint defects, chromosomal missegregation, cytokinesis failure, stimulation of chromosomal instability, anchorage-independent growth, and cell malignant transformation.⁵⁸ NLP co-localizes and interacts with BRCA1 at inter-phasic centrosome and thus the disruptions of BRCA1 function could affect NLP co-localization to centrosomes and induce the genomic instability.⁵⁹ NLP overexpression may also cause breast cancer resistance to paclitaxel chemotherapy.⁶⁰ In the EIF2 pathway, phosphorylation of eIF2u has been shown to play a role in maintaining normal cellular homeostasis and regulating cell growth,⁶² with dysregulation of EIF2 signaling pathway stimulating the cancerous tumors transformation.⁶³ In the valine, leucine and isoleucine biosynthesis, valine, leucine, and isoleucine are important branched-chain amino acids (BCAAs) for normal growth and development.⁶⁷ In the BCAA catabolism pathway, the first step is transamination, catalyzed by the branched chain amino acid transferase isozymes BCATs: a mitochondrial (BCATm) and a cytosolic (BCATc) isozyme.⁶⁸⁻⁷⁰ These five identified pathways can be used to identify ER+ breast cancer patients at risk of developing resistance to tamoxifen, and to identify treatment targets for such patients.

In conclusion, a systematic computational pathway-centric method identified molecular pathways to predict tamoxifen resistance. The identifies pathways and genes can be used to identify (i) cases at higher risk of developing resistance to tamoxifen that should be considered for alternative treatment manipulations (for instance, alternative endocrine therapy, radiation therapy, or chemotherapy, etc.) and (ii) cases who would benefit maximally from tamoxifen therapy.

### Example 1

### Materials and Methods

The Examples below were published in Rahem et al., EBioMedicine, 2020 November; 61:103047, herein incorporated by reference in its entirety.

### Patient Cohorts Utilized

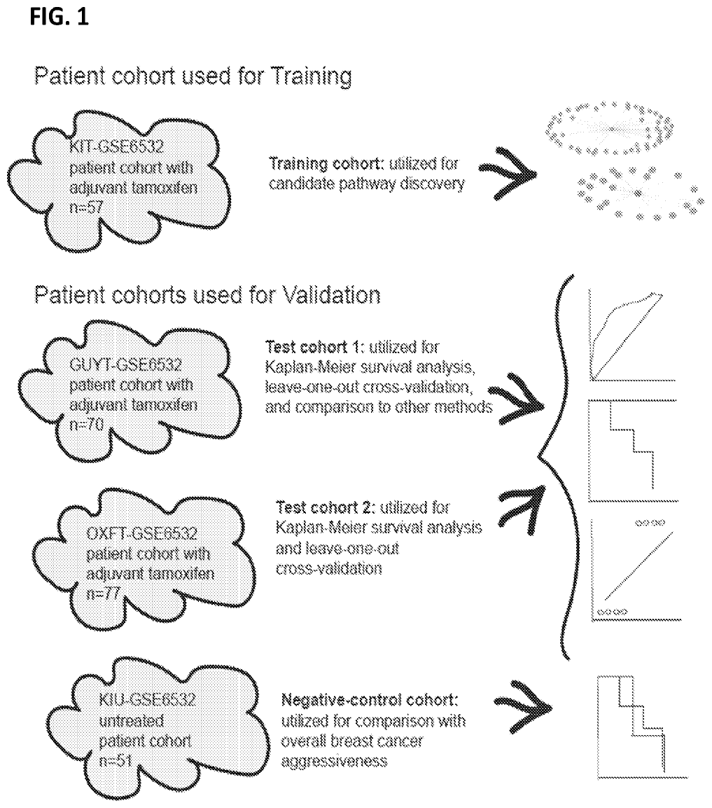

All gene expression datasets of patients with ER+ breast cancer were obtained from publicly available GEO data repository⁸⁴ from multi-institutional multi-PI comprehensive Loi et al.²⁹ study GSE6532 (FIG. 1, Table 2): (i) KIT-GSE6532 utilized as a Training cohort; (ii) GUYT-GSE6532, utilized as Test cohort 1; (iii) OXFT-GSE6532, utilized as a Test cohort 2; and (iv) KIU-GSE6532, utilized as a negative control cohort. Training cohort contains patient profiles of primary ER+ breast tumors (n=57), archived at the Uppsala University Hospital (Uppsala, Sweden), profiled on Affymetrix Human Genome U133A array and Affymetrix Human Genome U133B array. Test cohort 1 contains patient profiles of primary tumors from patients with ER+ breast cancer (n=70), archived at the Guy's Hospital (London, United Kingdom), profiled on Affymetrix Human Genome U133 Plus 2.0 Array. Test cohort 2 contains patient profiles of primary ER+ breast tumors (n=77), archived at the John Radcliffe Hospital (Oxford, United Kingdom), profiled on Affymetrix Human Genome U133A, B array. Negative control cohort consists of not-treated patients with ER+ primary breast tumors (n=51), profiled on Affymetrix Human Genome U133A, B array. All primary tumors samples in Training and Test cohorts collected through surgery, diagnosed between 1980 and 1995 and received tamoxifen-only treatment for 5 years post-diagnosis as their adjuvant treatment.

TABLE 2
Clinical characteristics of datasets used for Training and Testing analysis.

| | | | | Negative-control |
|---|---|---|---|---|
| | Training cohort | Test cohort 1 | Test cohort 2 | cohort |
| Characteristics | KIT-GSE6532 | GUYT-GSE6532 | OXFT-GSE6532 | KIU-GSE6532 |
| Platform | Affymetrix Human | Affymetrix Human | Affymetrix Human | Affymetrix Human |
| | Genome U133A, B | Genome U133 Plus | Genome U133A, B | Genome U133A, B |
| | array | 2.0 Array | array | array |
| Total number of | 57 | 70 | 77 | 51 |
| patients | | | | |
| PAM50 | | | | |
| Classification | | | | |
| Luminal A | 45 | 27 | 61 | 43 |
| Luminal B | 8 | 39 | 16 | 4 |
| *Other subtypes | 4 | 4 | — | 4 |
| Number of patients | 53 | 66 | 77 | 47 |
| utilized in our study | | | | |
| (other subtypes | | | | |
| excluded) | | | | |
| Number of events | 22/53 (41.5%) | 19/66 (28.78%) | 20/77 (25.97%) | 17/47 (36.17%) |
| Age | | | | |
| =<50 | 1 | 3 | 13 | 19 |
| >50 | 52 | 63 | 64 | 28 |
| Histological grade | | | | |
| 1 | 10 | 17 | 17 | 22 |
| 2 | 37 | 36 | 46 | 20 |
| 3 | 6 | 13 | 14 | 5 |
| Tumor size | | | | |
| ≤2 cm | 19 | 35 | 35 | 26 |
| >2 cm | 34 | 31 | 42 | 21 |
| Lymph node status | | | | |
| Negative | 14 | 2 | 48 | 47 |
| Positive | 39 | 44 | 29 | — |
| PR status | | | | |
| Negative | 4 | 16 | — | 1 |
| Positive | 49 | 50 | — | 46 |

Note:
*other subtypes = HER2-enriched, basal-like, or normal-like.

### Data Normalization

For each gene expression microarray dataset, matrix of RMA (Robust Microarray Analysis) normalized signal intensity values were used.⁴⁸ Using the current annotation file from GEO and the latest Affymetrix annotation files from Thermo Fisher database,⁸⁵ each probe set ID was annotated to gene ID, thereafter, probe IDs that annotated to different gene IDs or did not annotate to any gene ID were excluded. When multiple probe set IDs were mapped to the same gene, probes with the highest coefficient variation (CV) over all samples were selected. The CV for each probe was computed by dividing the standard deviation of expression values among all sample population by the mean expression value.^{86,87}

### Determining the Molecular Subtypes of Breast Cancer Patients

Gene expression classifier (PAM50) of the breast cancer subtypes was applied to assign BC patients to one of the intrinsic molecular subtypes: luminal A, luminal B, HER2-enriched, triple-negative/basal-like, and normal-like.^{32,88} The subtype classification of each patient was determined based on the closeness between the average expression profile of 50 genes in each subtype centroid and the corresponding gene expression pattern of patient tumor, where the distances measured utilizing Spearman's rank correlation.³² From genefu package in R, intrinsic.cluster.predict function with pam50⁸⁹ was utilized to eliminate samples with HER2-enriched, triple-negative/basal-like, and normal-like subtypes (i.e., non ER+).

Single-Sample Gene Set Enrichment Analysis (ssGSEA)

For single-sample analysis, gene expression values for each gene were transformed into standardized scores (i.e., z-scores) in order to bring the expression level into a common scale across all samples.^{36,90} Z-score for each gene was computed by subtracting the average intensity of the gene from the intensity of this gene in each sample and dividing it by the standard deviation (SD) across all samples.³⁶ In this way, each gene's mean is standardized to 0 and standard deviation to 1. Ranked list of z-scored for each gene for a given sample then defines a single-sample signature, utilized for further pathway enrichment analysis.

For pathway enrichment analysis, Reactome,³⁹ BioCarta⁴⁰ and KEGG⁴¹ databases were utilized, which contain 833 biological pathways, and implemented single-sample GSEA (i.e., ssGSEA^{37,38}), where each single-sample signature was used as a reference, and each pathway (i.e., genes from each pathway) was used as a query set. The GSEA normalized enrichment scores (NESs), and p-values were assessed utilizing 1,000 gene permutations. NES for each of the 833 pathways (i.e., also referred to as pathway activity levels) indicated how much each pathway is overrepresented in each single-sample signature. In particular, the positive NES would indicate a pathways enrichment in the top of the rank-ordered list (i.e., overexpressed part) of the signature and the negative NES would indicate pathway enrichment in the bottom of the rank ordered list (i.e., underexpressed part) of the signature.

Associating the Activity Levels of Molecular Pathways with Therapeutic Response

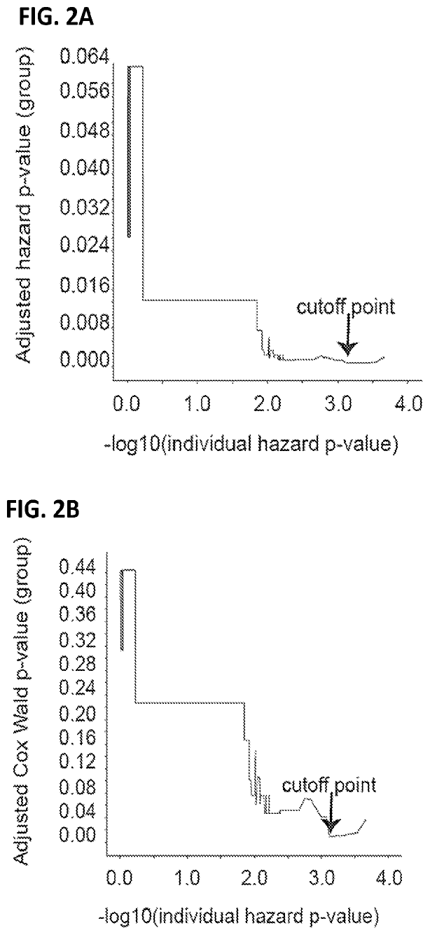

The activity levels of each pathway (i.e., NES) were then associated with tamoxifen response using Cox proportional hazards model,⁴² adjusted for common covariates, such as age, tumor grade, tumor size, lymph node status, and PR status. For this, R coxph function from survival package was utilized.⁹¹ To establish a robust threshold which should be utilized to select most significantly associated pathways, predictive ability of the pathways as a group (starting from the most significant pathway and then adding the next most significant pathway, one at a time) was evaluated. Thus, the groups of pathways that were evaluated were (i) Pathway 1; (ii) Pathways 1 and 2; (iii) Pathways 1, 2, and 3; etc. until all pathways were utilized. The predicted ability of each group was then evaluated and recorded in FIGS. 2A-2B. The cutoff point was determined as the one, where the addition of a pathway would not benefit an overall predictive ability of the group.

Furthermore, given that many of the 833 pathways exhibit parent-child relationships are heavily overlapping, all final pathways that had the above relationships were examined and if such dispute occurred, pathways with higher association with tamoxifen response were prioritized.

### Clinical Validation in Independent Patient Cohorts

For validation studies, the activity levels of the five pathways were used to stratify patients based on the risk of relapse due to treatment resistance in independent Test cohorts. Patient cohorts were subjected to t-SNE clustering, a widely-utilized dimensionality reduction technique,⁴⁴ using all pairs of high-dimensional (i.e., 5-dimensions in this study) points.^{45,92} In fact, t-SNE reduces high-dimensional dataset (i.e., 5-dimensional) in a low-dimensional (i.e., 2-dimensional) space and successfully distinguishes groups of patients that have similar pathway activity levels. Subsequently, k-means clustering⁴⁶ was utilized on t-SNE-derived the low-dimensional (i.e., 2-dimensional) space to obtain two groups of patients with distinct pathway activity patterns,^{45,92} using kmeans function in R.⁹³

The ability of the activity levels of the five molecular pathways to efficiently distinguish patient clusters was determined through receiver operating characteristics (ROC) analysis⁴⁷ on multiple (i.e., multivariable) logistic regression model, where normalized enrichment scores of 5 pathways were used as input parameters (i.e., independent/predictor variables) and patient clusters were utilized as a dependent/response variable. ROC curves were assessed using the area under the curve (AUC),⁴⁸ where AUC score of 0.5 indicates a random predictor. The logistic regression analysis was conducted using glm⁹⁴ function, and ROC analysis was performed using pROC⁹⁵ and ggplot2 packages in R.

Differences in therapeutic response between the patient groups were evaluated through Kaplan-Meier treatment-related survival analysis⁴⁹ and Cox proportional hazards model using survival and survminer packages⁴² in R. Log-rank p-value was utilized to assess the statistical significance of the Kaplan-Meier survival analysis and Wald p-value and hazard ratio were utilized for multivariable Cox proportional hazards model through survdiff and coxph functions from survival package.

To estimate the predictive accuracy of our model and obtain a more accurate indication of how well our finding behaves toward a new incoming patient, Leave-one-out cross-validation (LOOCV) was conducted.⁹⁶ In this method, one patient is “excluded/eliminated” and the rest of the patients are utilized for training purposes to the regression model. After that, a removed patient is assumed to be a new incoming patient and is assigned a risk of developing tamoxifen resistance. This process is repeated for each patient within a given dataset. LOOCV was implemented for multiple logistic regression model, where patient clusters membership was used as a response variable and normalized enrichment scores of the five pathways were utilized as input parameters. The logistic regression analysis was performed using glm⁹⁴ function, and LOOCV analysis was prepared using cv.glm function from boot package in R.

### Comparative Analysis to Other Commonly Utilized Approaches

To assess the superiority of our approach over other commonly used techniques, its performance was compared to (i) extreme-responder analysis;²⁷ (ii) SVM;³⁰ and (iii) PRES random forest.³¹ In each case, Training cohort for model training and Test cohort 1 for model validation was utilized. Groups of patients with poor and favorable response to tamoxifen in the Training cohort were compared by selecting: patients that experienced events within 1 year of tamoxifen administration (i.e., non-responders, n=4); and patients that did not experience any relapses for more than 9 years (i.e., responders, n=4) to define a differential expression signature of tamoxifen response (i.e., through two-sample two-tailed Welch t-test⁹⁷ through t.test function in R). For Epsi et al. method, the differential expression signature was then subjected to pathway enrichment analysis, where this signature was used as a reference and groups of genes from each pathway was used as a query gene set, and treated most significant pathways as candidate pathway markers. For SVM and PRES random forest, the differential expression signature (i.e., based on the proposed significance level) was subjected to the model training using Training cohort. The SVM analysis was performed using svm function from e1071 package, and PRES random forest analysis was prepared using train function from caret package in R. Predictive ability of the identified predictions was evaluated using Cox proportional hazards model through survival and survminer packages in R.

### Statistical Analysis

Statistical analysis was performed using R studio version 3.5.1 for statistical computing. For single-sample analysis, data were z-scored on individual gene level. For this, the mean and standard deviation was first estimated for each gene across all samples in the dataset. Subsequently, z-score for each gene was defined as the difference between its own intensity value and the mean of that gene across the samples and divided by the standard deviation for that gene. The ranked list of z-scores for each gene in a sample then defined single-sample signature. Pathway activity levels were estimated as Normalized Enrichment Scored (NESs) from the Gene Set Enrichment Analysis (GSEA), where NESs and p-values were estimated using 1,000 gene permutations. Cox proportional hazards model was utilized to associate pathway activity levels with treatment-related relapse-free survival (tRFS). When adjusting for common covariates multivariable Cox proportional hazards model was utilized, where its significance was reported using hazards ratio, hazards p-value, and Wald test. Kaplan-Meier survival analysis was utilized to estimate difference in treatment-related survival between two groups of patients, with log-rank p-value used to estimate significance. All survival analyses were subjected to adjustment for common covariates (e.g., tumor grade, tumor size, lymph node positivity, age, and PR negativity). Patients' cohorts were obtained from public repositories and all the code was assembles using available R packages, as described above, with no restrictions.

### Data Availability

Data utilized for Training and Testing and their clinical characteristics are available from GEO repository GSE6532.

### Example 2

### Training Phase: Identifying Molecular Pathways that Govern Primary Tamoxifen Resistance

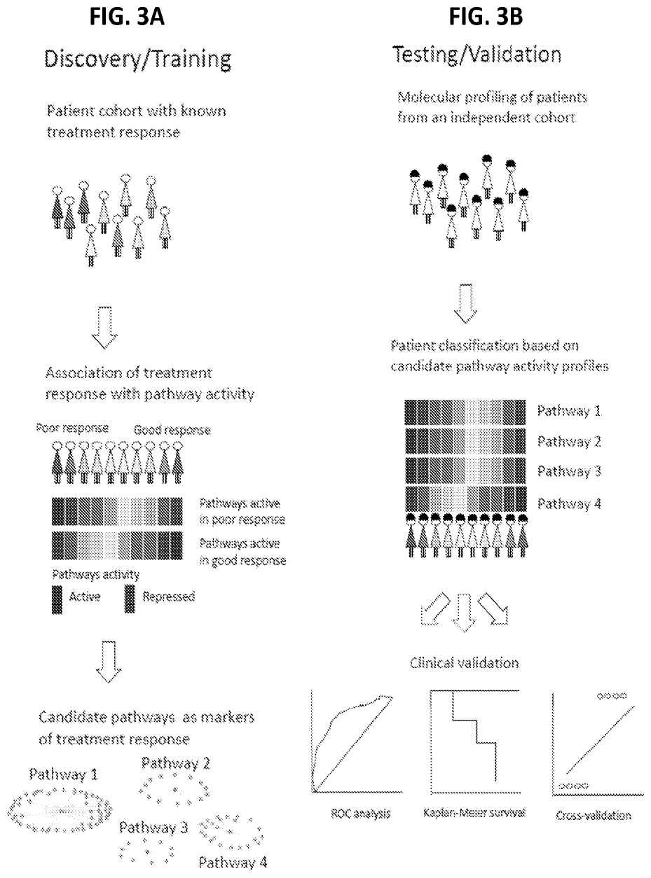

Provided is a genome-wide pathway-centric computational analysis to identify molecular pathways predictive of risk of resistance to tamoxifen in ER+ breast cancer patients. The approach included the following steps: Training phase (FIG. 3A): (i) activity levels of biological pathways is estimated in each ER+ breast cancer patient (across a wide spectrum of responses, present in a clinical setting) that received adjuvant tamoxifen (FIG. 1, Table 2); (ii) these pathway activity levels are then associated with tamoxifen treatment response across all patients, adjusted for common covariates; Testing phase (FIG. 3B): (iii) pathways that are significantly associated with the risk of tamoxifen resistance are then subjected to clinical validation analysis in independent patient cohorts (FIG. 1, Table 2), for their ability to predict tamoxifen resistance for new incoming patients; (iv) finally, ability of the five pathways to predict the risk of tamoxifen resistance is compared to other known gene signatures of resistance and overall disease aggressiveness, alongside comparison to other methods.

To accurately define therapeutic response to tamoxifen in ER+ breast cancer patients, gene expression profiles were carefully selected for the Training cohort (Loi et al.,²⁹ KIT-GSE6532) of primary ER+ breast tumors collected through surgery, not subjected to any neoadjuvant (i.e., prior to sample collection) treatment, and administered adjuvant (i.e., post-operative) 5-year long tamoxifen administration, with available clinical follow-up data (n=57) (FIG. 1, Table 2).

To avoid inconsistencies in BC classification, patient profiles of the Training cohort were subjected to a 50-gene Prediction Analysis of Microarrays panel³² (PAM50) classification. PAM50 classification categorized BC patients from the Training cohort into the five intrinsic molecular subtypes: luminal A, luminal B, human epidermal growth factor receptor 2 (HER2)-enriched, triple-negative/basal-like, and normal-like, known to differ in their clinical outcomes^{33,34} and therapy choice.³⁵ ER+BC, which is the phenotype of interest herein is contained within the luminal A and luminal B subtypes and is excluded from HER2-enriched, triple-negative/basal-like, and normal-like subtypes (Table 2). Out of 57 post-operative tamoxifen-treated patients, 4 patients were classified as HER2-enriched, basal-like, or normal-like, and thus were excluded from further analysis.

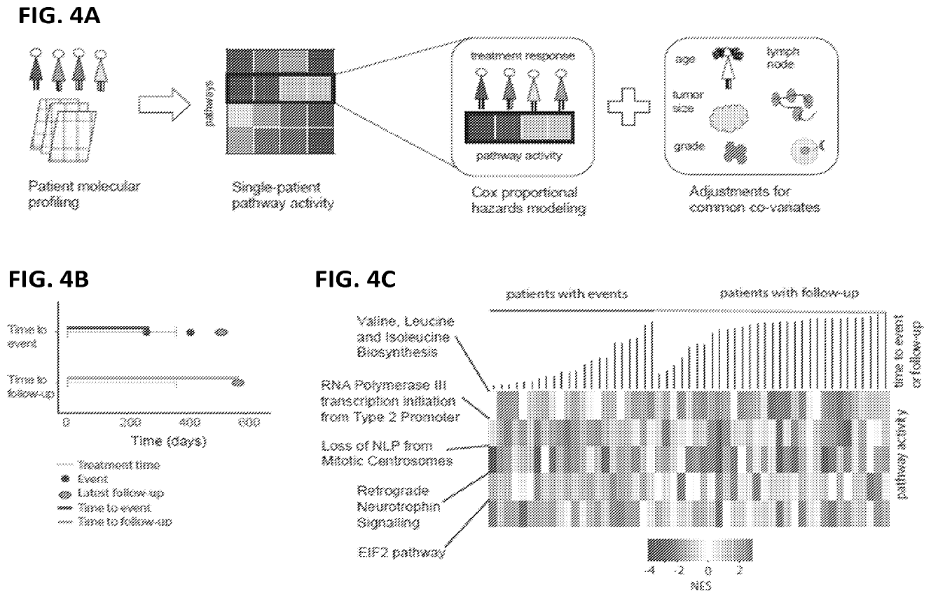

The objective was to evaluate tamoxifen response across all 53 patient samples (on the individual-patient level) and associate them with changes in biological pathway activities (FIG. 4a). To be able to evaluate each patient sample individually, gene expression profiles were scaled (i.e., z-scored, see Example 1) on individual gene levels so that each gene had mean 0 and standard deviation 1 over all samples in the Training cohort.³⁶ The list of genes ranked by their z-scores in each sample then defined an individual-patient signature. Each individual-patient signature was used to evaluate activity levels of biological pathways using single-sample Gene Set Enrichment Analysis (ssGSEA),^{37,38} where pathways were obtained from Reactome,³⁹ BioCarta⁴⁰ and KEGG⁴¹ databases, corresponding to 833 pathways. For this analysis, each patient signature was used as a reference and each pathway as a query gene set. Activity levels of biological pathways were defined by their enrichment in each patient signature, mathematically represented by the Normalized Enrichment Scores (NES) from the GSEA analysis, where positive NES corresponds to enrichment in the over-expressed part of the signature and negative NES corresponds to enrichment in the under-expressed part of the signature (FIGS. 3A, 4A).

Next, changes in pathway activity levels were associated to tamoxifen treatment response. In general, treatment-related relapse free survival (tRFS) was defined as the interval between tamoxifen administration (which occurred immediately after surgery) and the earliest relapse (defined as local, regional, or distant metastasis) or the latest follow-up (these patients did not develop an event until their latest follow-up). When a patient had a relapse during or after the therapy administration, time to therapy related relapse was defined from therapy start to the earliest relapse (FIG. 4B, top schematics, green line). When a patient never experienced a relapse, therapy-related relapse-free survival was measured from therapy start to the latest follow-up (FIG. 4B, bottom schematics, brown line). In this dataset, 41.5% of patients experienced tamoxifen-related events (i.e., relapse), making it ideally suited for Training purposes.

To estimate association between the activity levels of the biological pathways and tRFS across a wide spectrum of tamoxifen response (taking into account a heterogeneity of response to tamoxifen, present in a clinical setting), Cox proportional hazards model was utilized,⁴² ideally suited when time to event or follow-up is available. The Cox proportional hazards model was estimated between each pathway activity level (i.e., NESs, independent/predictor variable) and tamoxifen tRFS (i.e., dependent/response variable) across all 53 patients in the Training cohort. Furthermore, to account for the effect of other factors, this analysis was adjusted for commonly utilized covariates, as suggested in,⁴³ such as age, tumor grade, tumor size (>2 cm vs 5 2 cm), lymph node status, and PR status (note that decreased PR levels are associated with increased HER2 signaling¹⁶) (FIG. 4A). Such analysis identified five molecular pathways (FIG. 4C, Tables 1, 3), most significantly associated with response to tamoxifen (hazard p-value 5 0.00075, FIGS. 2A-2B), including Retrograde Neurotrophin Signalling, Loss of NLP from Mitotic Centrosomes, RNA Polymerase III Transcription Initiation from Type 2 Promoter, EIF2 pathway, and Valine Leucine and Isoleucine Biosynthesis, adjusting for parent-child relationships inherent in pathway databases (FIG. 5).

TABLE 3
Five molecular pathways and corresponding significance levels.

| | Adjusted Hazard | Adjusted |
|---|---|---|
| Pathway | ratio (95% CI) | Hazard p-value |
| REACTOME: RETROGRADE NEUROTROPHIN | 2.31(1.48-3.60) | 0.00021 |
| SIGNALLING | | |
| REACTOME: LOSS OF NLP FROM MITOTIC | 1.73(1.28-2.33) | 0.00029 |
| CENTROSOMES | | |
| REACTOME: RNA POLYMERASE III | 1.97(1.33-2.91) | 0.0006 |
| TRANSCRIPTION INITIATION FROM TYPE 2 | | |
| PROMOTER | | |
| BIOCARTA: EIF2 PATHWAY | 1.84(1.30-2.59) | 0.00053 |
| KEGG: VALINE LEUCINE AND ISOLEUCINE | 1.78(1.27- 2.50) | 0.00075 |
| BIOSYNTHESIS | | |

Note:
CI: confidence intervals.

### Example 3

### Testing Phase: Clinical Validation in Independent Patient Cohorts

The next step in the analysis was to evaluate the ability of the five pathways to predict treatment response to tamoxifen in independent non-overlapping clinical cohorts. For this, two patient cohorts were utilized for testing/validation purposes: (i) Test cohort 1²⁹ (GUYT-GSE6532, n=70) of primary breast tumors obtained at surgery, from patients that did not receive any neoadjuvant treatment and received only adjuvant tamoxifen, with 28.78% of patients having tamoxifen-related events (Table 2); and (ii) Test cohort 2²⁹ (OXFT-GSE6532, n=77) of primary breast tumors obtained at surgery, from patients that did not receive any neoadjuvant treatment and received only adjuvant tamoxifen, with 25.97% of patients with tamoxifen-related events. Both Test cohorts had clinical characteristics, neoadjuvant, and adjuvant conditions comparable to the Training cohort (Table 2). Similar to the analysis done on the Training cohort, AM50 classification was performed Pon the two Test cohorts, eliminating 4 patients from Test cohort 1 and keeping all patients for Test cohort 2.

The ability of the activity levels of the five pathways to predict risk of resistance to tamoxifen in two independent Test cohorts was evaluated. Activity levels for the five pathways in each patient was determined in the Test cohorts (similarly to Training cohorts, see Example 1) and subjected patients to t-distributed Stochastic Neighbor Embedding (t-SNE) clustering⁴⁴ as indicated in⁴⁵ for investigation of samples relationships. T-SNE analysis, which displays five-dimensional dataset in a two-dimensional space, stratified patients into two groups based on their pathway activity levels. The low-dimensional output (i.e., 2-dimensional) of t-SNE were then subjected to the k-means clustering⁴⁶ to correctly assign group membership (FIG. 6A for Test cohort 1 and FIG. 6D for Test cohort 2) one group with increased pathways' activities (orange) and one group with decreased pathways' activities (turquoise), mimicking the relationship that was observed in the Training cohort (FIG. 4C). The strength of group separation was confirmed through Receiver Operating Characteristic (ROC) analysis⁴⁷ using multiple logistic regression model (FIGS. 7A-7B), where normalized enrichment scores of the five pathways were used as input parameters (i.e., independent/predictor variables) and selected patient groups were utilized as a dependent/response variable. The efficiency of ROC analysis was estimated using area under the curve (AUC),⁴⁸ where AUC of 0.5 denotes a random predictor and AUC score of 1 denotes a perfect predictor (i.e., full separation of the patient groups). This analysis confirmed that the activity levels of the five pathways can be effectively used for classifying patients into distinct groups (Test cohort 1, AUC=0.929; Test cohort 2, AUC=0.867).

To assess if these patient groups significantly differ in their tamoxifen response, therapy-related relapse-free survivals was analyzed between the groups using Kaplan-Meier survival analysis⁴⁹ and Cox proportional hazards model,⁴² which demonstrated that the identified patient groups had a significant difference in their response to tamoxifen (Test cohort 1, log-rank p-value=0.02, FIG. 6B; Test cohort 2, log-rank p-value=0.01, FIG. 6E). These analyses were adjusted for common covariates⁴³ (i.e., age, tumor grade, tumor size, lymph node status, and PR status), demonstrating that these covariates did not significantly impact the predictive ability of the findings (Test cohort 1, adjusted hazard ratio=3.11, adjusted hazard p-value=0.044, 95% confidence interval CI: =1.03-9.396, FIG. 6B; Test cohort 2, adjusted hazard ratio=4.24, adjusted hazard p-value=0.012, CI: 1.3708-13.120, FIG. 6E).

Predictive accuracy of this model was examined in the two test cohorts using Leave-one-out cross-validation (LOOCV), which simulates a situation when a new incoming patient needs to be evaluated for her risks of developing resistance to tamoxifen. In particular, in LOOCV, one patient is “removed”, and the model is trained on the remaining patients, followed by the prediction of risk of resistance for the removed patient. The process is repeated for each patient. Using this analysis, the accurate performance of the model in predicting poor and favorable tamoxifen response for new incoming patients was demonstrated (Test cohort 1, accuracy for LOOCV=85.8%, FIG. 6C; Test cohort 2, accuracy for LOOCV=82.5%, FIG. 6F). Taken together, these findings indicate that the five pathway signature can predict patients at risk of tamoxifen resistance in independent patient cohorts.

### Example 4

### Comprehensive Comparison of Tamoxifen Response and Overall Disease Aggressiveness

A fundamental question in studying therapeutic response lies in its comparison to and differentiation from overall disease aggressiveness. The disclosed comprehensive investigation of this question was four-fold: (i) pathways implicated in disease aggressiveness were identified and compared their overlap with the five pathways of tamoxifen response; (ii) whether the five pathways can predict breast cancer aggressiveness in an independent (negative control) cohort was determined; (iii) the ability of the five pathways to predict tamoxifen response, given different status of PR receptor and Ki-67 proliferation index, which are known indicator of breast cancer aggressiveness was evaluated; and (iv) whether if known published signatures of disease aggressiveness could predict response to tamoxifen was evaluated.

First, to determine if the 5 pathways (FIG. 5, Table 3) overlap with pathways implicated in disease aggressiveness, a treatment-free prognostic pathway signature was developed using a patient cohort that received surgery only (KIU-GSE6532, n=51, negative control cohort).²⁹ Out of 51 surgery-treated patients, 4 patients were removed, based on the PAM50 classification. The single-sample pathway-based discovery approach was applied (as in the Training phase) and associated them to the RFS, which identified three pathways of aggressiveness (see Example 1) that showed no overlap with the five identified pathways, signifying that none of the five identified pathways are involved in cancer severity and are indeed specific to tamoxifen response.

Secondly, the ability of the five pathways to separate patients based on overall disease aggressiveness was evaluated if. Predictive ability of the five pathways on the BC patient cohort that did not receive any treatment after surgery (negative control cohort, as above) was analyzed. The dataset was subjected to the single-sample pathway enrichment analysis (for the five pathways, similarly to Test cohorts analysis). T-SNE clustering (FIG. 8A) and subsequent Kaplan-Meier survival analysis (FIG. 8B) on this cohort demonstrated that the five pathways do not separate patients base on their disease aggressiveness (hazard ratio=1.2, log-rank p-value=0.7, RFS was considered as a clinical endpoint), but rather specific for tamoxifen response. The effect of covariates (i.e., age, tumor grade, tumor size, and PR status) on disease progression in this setting was examined and observed that the five pathways remain insignificant, with tumor size significantly contributing to the disease progression (adjusted hazard p-value=0.0307).

Third, given that the PR status (which also reflects HER2 signaling) is an indicator of breast cancer aggressiveness, a stratified Kaplan-Meier analysis was performed on Test cohort 1 (for which this information was available). Test cohort 1 was divided into two groups: one with PR− positive status and one with PR-negative status. Both cohorts were separately subjected to t-SNE clustering, which demonstrated that the five pathways separated each cohort into patient sub-groups with high and low levels of pathway activities. Subsequent Kaplan-Meier survival analysis (FIGS. 9A, 9B, respectively) showed that these patient-subgroups significantly differ in their response to treatment (patients with PR-positive tumors, c-index=0.698, FIG. 9A; patients with PR-negative tumors, c-index=0.769, FIG. 9B), demonstrating that the five pathways can predict patients at risk of tamoxifen resistance regardless of the PR-status of the breast cancer. Similar analysis was performed on patients with different Ki-67 proliferation index (i.e., low levels of Ki-67 corresponding to Luminal A subtype and high levels of Ki-67 corresponding to Luminal B subtype) and demonstrated that the five pathways predict patients at risk of tamoxifen resistance independently of Ki-67 status (Luminal A/Ki-67 low, c-index=0.657, FIG. 9C; Luminal B/Ki-67 high, c-index=0.658, FIG. 9D).

Finally, to demonstrate that the predictive ability of the five pathways is not affected by other known markers of disease aggressiveness, whether known gene-based prognostic signatures can predict tamoxifen response or affect predictive ability of the five pathways was determined. For this, several known signatures of overall BC aggressiveness (i.e., prognostic signatures), including Wang et al. signature⁵⁰ (76 prognostic markers, with 57 present on U133 Plus 2.0) and van′t Veer et al. signature⁵¹ (70 prognostic markers, with 53 present on U133 Plus 2.0), were subjected to adjusted multivariable Cox proportional hazards model, alongside the five pathway signature, in the Test cohort 1. This analysis confirmed that the prognostic signatures were not predictive of tamoxifen response and did not impact predictive ability of the five pathways (adjusted hazard p-value=0.03, FIG. 8C).

Taken together, these findings indicate that the five-pathway signature of tamoxifen response is not indicative of overall breast cancer aggressiveness but is instead specific to response to tamoxifen.

### Example 5

### Comparative Analysis to Utilized Methods and Known Signatures of Tamoxifen Response

To evaluate predictive advantages of the five pathways, a comprehensive approach first (i) compared the predictive ability of the five pathways to predictions from other commonly used methods, including approaches based on extreme-responder analysis (i.e., tails of the distribution), support vector machine (SVM), and random forest; and second (ii) assessed if the predictive ability of the five pathways outperforms other known signatures of tamoxifen response.

Predictive ability of the five pathways was compared to predictions from other commonly utilized methods, such as (i) Epsi et al.²⁷, which utilized extreme-responder analysis, using tails of the treatment response distribution to define a treatment response signature; (ii) Zhong et al.³⁰, which used Support Vector Machine approach as a base; and (iii) Yu et al.³¹, also referred to as Personalized REgimen Selection (PRES), which used random forest approach as a base (see Example 1). To assure that all methods are comparable to the disclosed pathway-centric method, Epsi et al., Zhong et al., and Yu et al. methods were trained on the Training cohort, with each producing a list of predictions (predictions for Epsi et al.; 5 predictions for Zhong et al.; and 3 predictions for Yu et al.). These predictions were validated on the Test cohort 1, similarly to the disclosed pathway-centric method. Such analysis demonstrated that the five pathways outperform all three methods in their ability to predict the risk of tamoxifen treatment resistance (FIG. 10A: five pathways, hazard ratio=2.91, hazard p-value=0.031; Epsi et al., hazard ratio=2.79, hazard p-value=0.038; Zhong et al., hazard ratio=2.53, hazard p-value=0.063; Yu et al., hazard ratio=2.48, hazard p-value=0.058). Furthermore, these analyses were adjusted for the effect of common covariates (similarly to the original training phase), including age, tumor grade, tumor size, lymph node status and PR status and re-confirmed that the five pathways retain their significant predictive ability and outperform the other methods (FIG. 10B: five pathways, adjusted hazard ratio=3.11, adjusted hazard p-value=0.044; Epsi et al., adjusted hazard ratio=2.48, adjusted hazard p-value=0.076; Zhong et al., adjusted hazard ratio=2.96, adjusted hazard p-value=0.05; Yu et al., adjusted hazard ratio=2.81, adjusted hazard p-value=0.054).

To confirm that the predictive ability of the five pathways outperforms other known signatures in their ability to predict tamoxifen treatment response, known signature of tamoxifen response (i.e., predictive signatures), such as (i) Men et al.¹⁸ (10 predictive markers, with 9 present on U133 Plus 2.0); (ii) Paik et al.¹⁹ (also now as Oncotype DX, 21 predictive markers); and (iii) Ma et al.²⁰ (2 predictive markers) (FIG. 10C) were selected and used in adjusted multivariable Cox proportional hazards model, alongside the five pathway signature, utilizing Test cohort 1, as above. This analysis demonstrated that the additional predictive signatures do not significantly affect the ability of the five pathways to predict the risk of tamoxifen resistance (FIG. 10C, adjusted hazards p-value=0.03).

Taken together, these results demonstrate that the five pathway signature (FIG. 5, Tables 1 and 3) can be utilized to predict patients at risk of developing resistance to tamoxifen in a clinical setting and build a foundation for personalized therapeutic advice for patients with ER+ breast cancer.

### Example 6

### Pathway Read-Out Genes for Clinical Integration

Utilization of pathway activity levels in clinical setting might be hampered by the number of genes in each pathway and need for a full transcriptomic profiling of patients, which might be both time- and cost-sensitive. To address these issues and bring the model closer to clinical utilization, “read-out” genes were identified for each pathway. Expression of these genes would (i) accurately reflect pathway activity levels (i.e., through Spearman correlation between gene expression levels and pathway activity levels) and (ii) be significantly associated with treatment response (i.e., through adjusted Cox proportional hazards); thus making them suitable as marker read-outs for tamoxifen resistance.

Using such analyses, five read-out genes (one for each pathway, FIG. 11) were identified in Training cohort and it was demonstrated that they are equally effective (compared to the five pathways) in predicting tamoxifen response in both Test cohort 1 and Test cohort 2, making them suitable candidates for clinical integration. These five read-out genes included (i) AP2S1 (Retrograde Neurotrophin Signalling pathway), (ii) CDC2 (Loss of NLP from Mitotic Centrosomes pathway), (iii) GTF3C3 (RNA Polymerase III Transcription Initiation from Type 2 Promoter pathway), (iv) EIF2AK3 (EIF2 pathway), and (v) LARS (Valine Leucine and Isoleucine Biosynthesis pathway).

Read-out genes were associated with tamoxifen sensitivity in human cancer cell lines by performing cancer dependency map analysis using DepMap web portal (depmap portal, Explore the Cancer Dependency Map, https://depmap.org/portal/(2019)), which utilizes PRISM Repurposing (Corsello et al. (2019) bioRxiv, https://doi.org/10.1101/730119, CTD2 (Seashore-Ludlow et al., Cancer Discov. (2015) 5:1210-23) (Rees et al., Nat. Chem. Biology (2016) 12:109-16), and GDSC databases (Iorio et al., Cell (2016) 166:740-54). Dependency map screened for sensitivity to multiple anti-cancer drugs (including tamoxifen) across various human cancer cell lines. Measures of dose response were obtained using the area under the dose-response curve (AUCs) scores for each drug-cancer cell lines pair where large AUC scores show decreased sensitivity to the drug and small scores show increased sensitivity to the drug. mRNA expression of identified read-out genes was used to query this resource where large AUCs showed poor or no response to tamoxifen and smaller AUCs values showed favorable response. Overall, this analysis showed decreased sensitivity to tamoxifen treatment in different human cancer cell lines (high AUCs), based on the expression levels of the read-out genes in these cell lines, which is consistent with conclusions made herein.

To further the utilization of such read-out genes into the clinical setting, expression levels were used to define a risk score to develop tamoxifen resistance. For this, a ROC analysis was performed for each read-out gene in the Training cohort, which reflected each gene's ability to separate patients into good and poor response groups. Ranks of these ROC scores from the Training cohort were then utilized as weights for each read-out gene, so that the risk score of tamoxifen resistance was defined as the weighted sum of expression values for read-out genes (where expression values were multiplied by the weights corresponding to the ranks of ROC values) (FIG. 11A). The read out genes (one per pathway) were then used to define a risk to develop resistance to tamoxifen. The risk score was calculated as a weighted sum of the read-out genes expression values, multiplied by their ROC value ranks (which defined their ability to differentiate patients with good and poor response in a Training set) as

$$risk score = {\sum }_{k = 1}^{\# of read out genes} x ( k ) * w ( k ) \qquad ( Equation 1 )$$

where k is a read-out gene, x(k) is expression value of k, and w(k) is a weight (i.e., ROC value rank) for k. The risk scores were then separated into low/intermediate risk (≤mean+1SD) and high risk (>mean+1SD) groups, which were further evaluated using Kaplan-Meier survival analysis and Cox proportional hazards model. The weight for each gene is shown below. Thus, for example, the expression value obtained for CDC2 was multiplied by 3, the expression value obtained for LARS was multiplied by 2, the expression value obtained for GTF3C3 was multiplied by 1, the expression value obtained for AP2S1 was multiplied by 1, and the expression value obtained for EIF2AK3 was multiplied by 1, and these five values summed.

| Gene name | AUC values | Weight |
|---|---|---|
| CDC2 | 0.8016 | 3 |
| LARS | 0.7717 | 2 |
| GTF3C3 | 0.7364 | 1 |
| AP2S1 | 0.7283 | 1 |
| EIF2AK3 | 0.7242 | 1 |

The risk scores were defined for both Test cohort 1 and Test cohort 2 and risk score distribution defined high risk (>mean+1SD, where mean+1SD for both cohorts were equal to 4.5 score) and low/intermediate risk (≤mean+1SD) patients (FIGS. 111B, 11D). Such groups were then subjected Kaplan-Meier survival analysis (FIGS. 11C, 11E), which demonstrated that risk scores based on read-out genes are equally effective (compared to activity levels of five candidate pathways) in predicting tamoxifen response in both Test cohort 1 and Test cohort 2, making them suitable candidates for clinical integration.

In summary, provided is a systematic computational pathway-centric method that identified molecular pathways and their read-out genes to predict tamoxifen resistance. The disclosed methods can be used to prioritize and determine (i) cases at higher risk of developing resistance to tamoxifen that should be considered for alternative treatment manipulations (for instance, alternative endocrine therapy, radiation therapy, or chemotherapy etc.) and (ii) cases who would benefit maximally from tamoxifen therapy.

### REFERENCES

1 Zhang et al., Estrogen receptor-positive breast cancer molecular signatures and therapeutic potentials (Review). Biomed Rep 2, 41-52, doi:10.3892/br.2013.187 (2014).
2 Pedraza, V. et al. Gene expression signatures in breast cancer distinguish phenotype characteristics, histologic subtypes, and tumor invasiveness. Cancer 116, 486-496, doi:10.1002/cncr.24805 (2010).
3 Siegel et al., Cancer statistics, 2019. CA: a cancer journal for clinicians 69, 7-34 (2019).
4 Chang, M. Tamoxifen resistance in breast cancer. Biomol Ther (Seoul) 20, 256-267, doi:10.4062/biomolther.2012.20.3.256 (2012).
5 Hayes, E. L. & Lewis-Wambi, J. S. Mechanisms of endocrine resistance in breast cancer: an overview of the proposed roles of noncoding RNA. Breast Cancer Res 17, 40, doi:10.1186/s13058-015-0542-y (2015).
6 Group, E. B. C. T. C. Tamoxifen for early breast cancer: an overview of the randomised trials. The Lancet 351, 1451-1467 (1998).
7 Hackshaw, A. et al. Long-term benefits of 5 years of tamoxifen: 10-year follow-up of a large randomized trial in women at least 50 years of age with early breast cancer. J Clin Oncol 29, 1657-1663 (2011).
8 Davies, C. et al. Early Breast Cancer Trialists' Collaborative G. Relevance of breast cancer hormone receptors and other factors to the efficacy of adjuvant tamoxifen: patient-level meta-analysis of randomised trials. Lancet 378, 771-784 (2011).
9 Davies, C. et al. Long-term effects of continuing adjuvant tamoxifen to 10 years versus stopping at 5 years after diagnosis of oestrogen receptor-positive breast cancer: ATLAS, a randomised trial. The Lancet 381, 805-816 (2013).
10 Loi, S. et al. Predicting prognosis using molecular profiling in estrogen receptor-positive breast cancer treated with tamoxifen. BMC Genomics 9, 239, doi:10.1186/1471-2164-9-239 (2008).
11 Gallo, M. A. & Kaufman, D. in Seminars in oncology. S1-71-S71-80.
12 Fox et al., Abrogating endocrine resistance by targeting ERalpha and PI3K in breast cancer. Front Oncol 2, 145, doi:10.3389/fonc.2012.00145 (2012).
13 Osborne, C. K. Tamoxifen in the treatment of breast cancer. New England Journal of Medicine 339, 1609-1618 (1998).
14 Shou, J. et al. Mechanisms of tamoxifen resistance: increased estrogen receptor-HER2/neu cross-talk in ER/HER2-positive breast cancer. J Natl Cancer Inst 96, 926-935 (2004).
15 Osborne, C. K. et al. Role of the estrogen receptor coactivator AIB1 (SRC-3) and HER-2/neu in tamoxifen resistance in breast cancer. J Natl Cancer Inst 95, 353-361 (2003).
16 Cui et al., Biology of progesterone receptor loss in breast cancer and its implications for endocrine therapy. Journal of clinical oncology 23, 7721-7735 (2005).
17 Dowsett, M. et al. Relationship between quantitative estrogen and progesterone receptor expression and human epidermal growth factor receptor 2 (HER-2) status with recurrence in the Arimidex, Tamoxifen, Alone or in Combination trial. Journal of clinical oncology 26, 1059-1065 (2008).
18 Men, X. et al. Transcriptome profiling identified differentially expressed genes and pathways associated with tamoxifen resistance in human breast cancer. Oncotarget 9, 4074-4089, doi:10.18632/oncotarget.23694 (2018).
19 Paik, S. et al. A multigene assay to predict recurrence of tamoxifen-treated, node-negative breast cancer. N Engl J Med 351, 2817-2826, doi:10.1056/NEJMoa041588 (2004).
20 Ma, X.-J. et al. A two-gene expression ratio predicts clinical outcome in breast cancer patients treated with tamoxifen. Cancer cell 5, 607-616 (2004).
21 Chen et al., Molecular signature of cancer at gene level or pathway level? Case studies of colorectal cancer and prostate cancer microarray data. Computational and mathematical methods in medicine 2013 (2013).
22 Wang, Y. et al. Identifying novel prostate cancer associated pathways based on integrative microarray data analysis. Computational biology and chemistry 35, 151-158 (2011).
23 Myers, J. S., von Lersner, A. K., Robbins, C. J. & Sang, Q.-X. A. Differentially expressed genes and signature pathways of human prostate cancer. PloS one 10, e0145322 (2015).
24 Abraham, G., Kowalczyk, A., Loi, S., Haviv, I. & Zobel, J. Prediction of breast cancer prognosis using gene set statistics provides signature stability and biological context. BMC bioinformatics 11, 277 (2010).
25 Tian, L. et al. Discovering statistically significant pathways in expression profiling studies. Proceedings of the National Academy of Sciences 102, 13544-13549 (2005).
26 Lee, E., Chuang, H.-Y., Kim, J.-W., Ideker, T. & Lee, D. Inferring pathway activity toward precise disease classification. PLoS computational biology 4, e1000217 (2008).
27 Chuang, H. Y., Lee, E., Liu, Y. T., Lee, D. & Ideker, T. Network-based classification of breast cancer metastasis. Molecular systems biology 3, 140 (2007).
28 Epsi, N. J., Panja, S. & Pine, S. R. pathCHEMO, a generalizable computational framework uncovers molecular pathways of chemoresistance in lung adenocarcinoma. 2, 334, doi:10.1038/s42003-019-0572-6 (2019).
29 Loi, S. et al. Definition of clinically distinct molecular subtypes in estrogen receptor-positive breast carcinomas through genomic grade. Journal of clinical oncology 25, 1239 (2007).
30 Zhong, Q. et al. A response prediction model for taxane, cisplatin, and 5-fluorouracil chemotherapy in hypopharyngeal carcinoma. Scientific reports 8, 12675 (2018).
31 Yu, K. et al. Personalized chemotherapy selection for breast cancer using gene expression profiles. Scientific reports 7, 43294 (2017).
32 Parker, J. S. et al. Supervised risk predictor of breast cancer based on intrinsic subtypes. J Clin Oncol 27, 1160-1167, doi:10.1200/jco.2008.18.1370 (2009).
33 Sorlie, T. et al. Gene expression patterns of breast carcinomas distinguish tumor subclasses with clinical implications. Proc Natl Acad Sci USA 98, 10869-10874, doi:10.1073/pnas.191367098 (2001).
34 Hu, Z. et al. The molecular portraits of breast tumors are conserved across microarray platforms. BMC Genomics 7, 96, doi:10.1186/1471-2164-7-96 (2006).
35 Rouzier, R. et al. Nomograms to predict pathologic complete response and metastasis-free survival after preoperative chemotherapy for breast cancer. J Clin Oncol 23, 8331-8339, doi:10.1200/jco.2005.01.2898 (2005).
36 Cheadle, C., Vawter, M. P., Freed, W. J. & Becker, K. G. Analysis of microarray data using Z score transformation. J Mol Diagn 5, 73-81, doi:10.1016/s1525-1578(10)60455-2 (2003).
37 Subramanian, A. et al. Gene set enrichment analysis: a knowledge-based approach for interpreting genome-wide expression profiles. Proc Natl Acad Sci USA 102, 15545-15550, doi:10.1073/pnas.0506580102 (2005).
38 Barbie, D. A. et al. Systematic RNA interference reveals that oncogenic KRAS-driven cancers require TBK1. Nature 462, 108-112, doi:10.1038/nature08460 (2009).
39 Fabregat, A. et al. The reactome pathway knowledgebase. Nucleic acids research 44, D481-D487 (2015).
Pandey, R., Guru, R. K. & Mount, D. W. Pathway Miner: extracting gene association networks from molecular pathways for predicting the biological significance of gene expression microarray data. Bioinformatics 20, 2156-2158 (2004).
41 Ogata, H. et al. KEGG: Kyoto encyclopedia of genes and genomes. Nucleic acids research 27, 29-34 (1999).
42 Cox, D. R. Regression models and life-tables. Journal of the Royal Statistical Society: Series B (Methodological) 34, 187-202 (1972).
43 Cianfrocca, M. & Goldstein, L. J. Prognostic and predictive factors in early-stage breast cancer. Oncologist 9, 606-616, doi:10.1634/theoncologist.9-6-606 (2004).
44 Maaten, L. v. d. & Hinton, G. Visualizing data using t-SNE. Journal of machine learning research 9, 2579-2605 (2008).
Taskesen, E. & Reinders, M. J. 2D Representation of Transcriptomes by t-SNE Exposes Relatedness between Human Tissues. PLoS One 11, e0149853, doi:10.1371/journal.pone.0149853 (2016).
46 Hartigan, J. A. & Wong, M. A. Algorithm AS 136: A k-means clustering algorithm. Journal of the Royal Statistical Society. Series C (Applied Statistics) 28, 100-108 (1979).
47 Hajian-Tilaki, K. Receiver operating characteristic (ROC) curve analysis for medical diagnostic test evaluation. Caspian journal of internal medicine 4, 627 (2013).
48 Hanley, J. A. & McNeil, B. J. The meaning and use of the area under a receiver operating characteristic (ROC) curve. Radiology 143, 29-36 (1982).
49 Goel, M. K., Khanna, P. & Kishore, J. Understanding survival analysis: Kaplan-Meier estimate. Int J Ayurveda Res 1, 274-278, doi:10.4103/0974-7788.76794 (2010).
50 Wang, Y. et al. Gene-expression profiles to predict distant metastasis of lymph-node-negative primary breast cancer. Lancet 365, 671-679, doi:10.1016/s0140-6736(05)17947-1 (2005).
51 van't Veer, L. J. et al. Gene expression profiling predicts clinical outcome of breast cancer. Nature 415, 530-536, doi:10.1038/415530a (2002).
52 Menashe, I. et al. Large-scale pathway-based analysis of bladder cancer genome-wide association data from five studies of European background. PloS one 7, e29396 (2012).
53 Kim, J. et al. NTRK1 fusion in glioblastoma multiforme. PLoS One 9, e91940 (2014).
54 Martin-Zanca, D., Hughes, S. H. & Barbacid, M. A human oncogene formed by the fusion of truncated tropomyosin and protein tyrosine kinase sequences. Nature 319, 743 (1986).
55 Greco, A. et al. TRK-T1 is a novel oncogene formed by the fusion of TPR and TRK genes in human papillary thyroid carcinomas. Oncogene 7, 237-242 (1992).
56 Vaishnavi, A. et al. Oncogenic and drug-sensitive NTRK1 rearrangements in lung cancer. Nature medicine 19, 1469 (2013).
57 Vaishnavi, A., Le, A. T. & Doebele, R. C. TRKing down an old oncogene in a new era of targeted therapy. Cancer discovery 5, 25-34 (2015).
58 Li, J. & Zhan, Q. The role of centrosomal Nlp in the control of mitotic progression and tumourigenesis. British journal of cancer 104, 1523 (2011).
59 Jin, S. et al. BRCA1 interaction of centrosomal protein Nlp is required for successful mitotic progression. Journal of Biological Chemistry 284, 22970-22977 (2009).
60 Zhao, W., Song, Y., Xu, B. & Zhan, Q. Overexpression of centrosomal protein Nlp confers breast carcinoma resistance to paclitaxel. Cancer biology & therapy 13, 156-163 (2012).
61 Strebhardt, K. & Ullrich, A. Targeting polo-like kinase 1 for cancer therapy. Nature reviews cancer 6, 321 (2006).
62 Burwick, N. & Aktas, B. H. The eIF2-alpha kinase HRI: A potential target beyond the red blood cell. Expert opinion on therapeutic targets 21, 1171-1177 (2017).
63 Donze et al., Abrogation of translation initiation factor eIF-2 phosphorylation causes malignant transformation of NIH 3T3 cells. The EMBO Journal 14, 3828-3834 (1995).
64 Lobo, M. V. et al. Levels, phosphorylation status and cellular localization of translational factor eIF2 in gastrointestinal carcinomas. The Histochemical Journal 32, 139-150 (2000).
65 Wang, S. et al. Expression of the eukaryotic translation initiation factors 4E and 2α in non-Hodgkin's lymphomas. The American journal of pathology 155, 247-255 (1999).
66 Burwick, N. et al. The eIF2-alpha kinase HRI is a novel therapeutic target in multiple myeloma. Leukemia research 55, 23-32 (2017).
67 Hutson, S. M., Sweatt, A. J. & LaNoue, K. F. Branched-chain amino acid metabolism: implications for establishing safe intakes. The Journal of nutrition 135, 15575-15645 (2005).
68 Hutson et al., Role of mitochondrial transamination in branched chain amino acid metabolism. Journal of Biological Chemistry 263, 3618-3625 (1988).
69 Wallin, R., Hall, T. R. & Hutson, S. M. Purification of branched chain aminotransferase from rat heart mitochondria. Journal of Biological Chemistry 265, 6019-6024 (1990).
70 Hall, T., Wallin, R., Reinhart, G. & Hutson, S. Branched chain aminotransferase isoenzymes. Purification and characterization of the rat brain isoenzyme. Journal of Biological Chemistry 268, 3092-3098 (1993).
71 Mayers, J. R. et al. Tissue of origin dictates branched-chain amino acid metabolism in mutant Kras-driven cancers. Science 353, 1161-1165 (2016).
72 Dey, P. et al. Genomic deletion of malic enzyme 2 confers collateral lethality in pancreatic cancer. Nature 542, 119 (2017).
73 Tönjes, M. et al. BCAT1 promotes cell proliferation through amino acid catabolism in gliomas carrying wild-type IDH1. Nature medicine 19, 901 (2013).
74 Zhou, W. et al. Over-expression of BCAT1, a c-Myc target gene, induces cell proliferation, migration and invasion in nasopharyngeal carcinoma. Molecular cancer 12, 53 (2013).
75 Sharma, D. et al. Release of methyl CpG binding proteins and histone deacetylase 1 from the estrogen receptor α (ER) promoter upon reactivation in ER-negative human breast cancer cells. Molecular endocrinology 19, 1740-1751 (2005).
76 Jin, S. et al. A network-based approach to uncover microRNA-mediated disease comorbidities and potential pathobiological implications. npj Systems Biology and Applications 5, 41, doi:10.1038/s41540-019-0115-2 (2019).
77 Braga, T. V. et al. Evaluation of MiR-15a and MiR-16-1 as prognostic biomarkers in chronic lymphocytic leukemia. Biomedicine & pharmacotherapy=Biomedecine & pharmacotherapie 92, 864-869, doi:10.1016/j.biopha.2017.05.144 (2017).
78 Bandi, N. et al. miR-15a and miR-16 are implicated in cell cycle regulation in a Rb-dependent manner and are frequently deleted or down-regulated in non-small cell lung cancer. Cancer research 69, 5553-5559, doi:10.1158/0008-5472.can-08-4277 (2009).
79 Cava, C. et al. How interacting pathways are regulated by miRNAs in breast cancer subtypes. BMC bioinformatics 17, 348 (2016).
80 Miller, T. E. et al. MicroRNA-221/222 confers tamoxifen resistance in breast cancer by targeting p27Kip1. Journal of biological chemistry 283, 29897-29903 (2008).
81 Cimino, D. et al. miR148b is a major coordinator of breast cancer progression in a relapse-associated microRNA signature by targeting ITGAS, ROCK1, PIK3CA, NRAS, and CSF1. The FASEB Journal 27, 1223-1235 (2013).
82 Sun, Z. et al. Effect of exosomal miRNA on cancer biology and clinical applications. Molecular cancer 17, 147 (2018).
83 Gawel et al. A validated single-cell-based strategy to identify diagnostic and therapeutic targets in complex diseases. Genome medicine 11, 47, doi:10.1186/s13073-019-0657-3 (2019).
84 Barrett, T. et al. NCBI GEO: archive for functional genomics data sets—update. Nucleic Acids Res 41, D991-995, doi:10.1093/nar/gks1193 (2013).
85 ThermoFisher Scientific. Human genome u133 set—support materials, <www.affymetrix.com/support/technical/byproduct.affx?product=hgu133>(2018, May 25).
86 Negi, S. K. & Guda, C. Global gene expression profiling of healthy human brain and its application in studying neurological disorders. Sci Rep 7, 897, doi:10.1038/s41598-017-00952-9 (2017).
87 Arnatkevic et al., A practical guide to linking brain-wide gene expression and neuroimaging data. Neuroimage 189, 353-367, doi:10.1016/j.neuroimage.2019.01.011 (2019).
88 Chia, S. K. et al. A 50-gene intrinsic subtype classifier for prognosis and prediction of benefit from adjuvant tamoxifen. Clin Cancer Res 18, 4465-4472, doi:10.1158/1078-0432.ccr-12-0286 (2012).
89 Haibe-Kains et al., J. genefu: Relevant functions for gene expression analysis, especially in breast cancer. R/Bioconductor. version: Development (2.12) (2011).
90 Lee et al., Inferring pathway activity toward precise disease classification. PLoS Comput Biol 4, e1000217, doi:10.1371/journal.pcbi.1000217 (2008).
91 Therneau, T. M. & Grambsch, P. M. Modeling survival data: extending the Cox model. (Springer Science & Business Media, 2013).
92 Mwangi, B., Soares, J. C. & Hasan, K. M. Visualization and unsupervised predictive clustering of high-dimensional multimodal neuroimaging data. J Neurosci Methods 236, 19-25, doi:10.1016/j.jneumeth.2014.08.001 (2014).
93 stat. K-means clustering, <https://stat.ethz.ch/R-manual/R-devel/library/stats/html/kmeans.html> (2019, Mar. 21).
94 Zeileis et al., Regression models for count data in R. Journal of statistical software 27, 1-25 (2008).
95 Robin, X. et al. pROC: an open-source package for R and S+ to analyze and compare ROC curves. BMC bioinformatics 12, 77 (2011).
96 Mosteller, F. & Tukey, J. W. Data analysis, including statistics. Handbook of social psychology 2, 80-203 (1968).
97 Welch, B. L. The generalization of student's′ problem when several different population variances are involved. Biometrika 34, 28-35 (1947).

In view of the many possible embodiments to which the principles of the disclosure may be applied, it should be recognized that the illustrated embodiments are only examples of the disclosure and should not be taken as limiting in scope. Rather, the scope of the invention is defined by the following claims. We, therefore, claim as our invention all that comes within the scope and spirit of these claims.

We claim:

1. A method of treating a subject with an estrogen receptor positive (ER+) breast cancer, comprising:
(i) measuring expression of ER+ breast cancer-related molecules from ER+ breast cancer-related pathways in a sample obtained from a subject with ER+ breast cancer, wherein the ER+ breast cancer-related pathways comprise retrograde neurotrophin signalling, loss of NLP from mitotic centrosomes, RNA polymerase III transcription initiation from Type 2 promoter, EIF2 pathway, and valine, leucine and isoleucine biosynthesis; and
(ii) administering a therapeutically effective amount of tamoxifen to the subject, thereby treating the subject with ER+ breast cancer, wherein expression of the ER+ breast cancer-related molecules is decreased relative to a control representing expression of the ER+ breast cancer-related molecules expected in a sample from a ER+ breast cancer that develops resistance to the tamoxifen; or
administering a therapeutically effective amount of a non-tamoxifen therapy to the subject, thereby treating the subject with ER+ breast cancer, wherein expression of the ER+ breast cancer-related molecules is increased relative to a control representing expression of the ER+ breast cancer-related molecules expected in a sample from a ER+ breast cancer that does not develop resistance to the tamoxifen.

2. A method of identifying a subject with ER+ breast cancer who will not develop resistance to tamoxifen, comprising:
measuring expression of ER+ breast cancer-related molecules from ER+ breast cancer-related pathways in a sample obtained from a subject with ER+ breast cancer, wherein the ER+ breast cancer-related pathways comprise retrograde neurotrophin signalling, loss of NLP from mitotic centrosomes, RNA polymerase III transcription initiation from Type 2 promoter, EIF2 pathway, and valine, leucine and isoleucine biosynthesis; and
comparing expression of the ER+ breast cancer-related molecules from ER+ breast cancer-related pathways to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen;
wherein:
expression of the ER+ breast cancer-related molecules is decreased relative to the control representing expression for the ER+ breast cancer-related molecules expected in a sample from a subject who will develop resistance to tamoxifen, or is similar to the control representing expression for the ER+ breast cancer-related molecules expected in a sample from a subject who will not develop resistance to tamoxifen,
thereby identifying a subject with ER+ breast cancer who will not develop resistance to tamoxifen.

3. A method of identifying a subject with ER+ breast cancer who will develop resistance to tamoxifen, comprising:
measuring expression of ER+ breast cancer-related molecules from ER+ breast cancer-related pathways in a sample obtained from a subject with ER+ breast cancer, wherein the ER+ breast cancer-related pathways comprise retrograde neurotrophin signalling, loss of NLP from mitotic centrosomes, RNA polymerase III transcription initiation from Type 2 promoter, EIF2 pathway, and valine, leucine and isoleucine biosynthesis; and
comparing expression of the ER+ breast cancer-related molecules from ER+ breast cancer-related pathways to a control representing expression for the ER+ breast cancer-related molecules expected in a sample from a subject who will develop resistance to tamoxifen, or to a control representing expression for the ER+ breast cancer-related molecules expected in a sample from a subject who will not develop resistance to tamoxifen;
wherein:
expression of the ER+ breast cancer-related molecules is similar to the control representing expression for the ER+ breast cancer-related molecules expected in a sample from a subject who will develop resistance to tamoxifen, or is increased relative to the control representing expression for the ER+ breast cancer-related molecules expected in a sample from a subject who will not develop resistance to tamoxifen,
thereby identifying a subject with ER+ breast cancer who will develop resistance to tamoxifen.

4. The method of any one of claims 1-3, wherein the ER+ breast cancer-related molecules from the ER+ breast cancer-related pathways comprise:
adaptor-related protein complex 2, sigma-1 subunit (AP2S1) from the retrograde neurotrophin signaling pathway,
cyclin-dependent kinase 2 (CDC2) from the loss of NLP from mitotic centrosomes pathway,
general transcription factor IIIC subunit 3 (GTF3C3) from the RNA polymerase III transcription initiation from Type 2 promoter pathway,
eukaryotic translation initiation factor 2-alpha kinase 3 (EIFA2AK3) from the EIF2 pathway, and
leucyl-tRNA synthetase (LARS) from the valine, leucine and isoleucine biosynthesis pathway.

5. The method of claim 4, wherein expression of AP2S1 is determined by measuring expression of a nucleic acid molecule comprising at least 80% sequence identity to SEQ ID NO: 1; expression of CDC2 is determined by measuring expression of a nucleic acid molecule comprising at least 80% sequence identity to SEQ ID NO: 2, expression of EIFA2AK3 is determined by measuring expression of a nucleic acid molecule comprising at least 80% sequence identity to SEQ ID NO: 3 expression of GTF3C3 is determined by measuring expression of a nucleic acid molecule comprising at least 80% sequence identity to SEQ ID NO: 4, and/or expression of LARS is determined by measuring expression of a nucleic acid molecule comprising at least 80% sequence identity to SEQ ID NO: 5.

6. The method of claim 4 or 5, wherein expression of AP2S1 is determined by measuring expression of a protein encoded by a sequence comprising at least 80% sequence identity to SEQ ID NO: 1; expression of CDC2 is determined by measuring expression of a protein encoded by a sequence comprising at least 80% sequence identity to SEQ ID NO: 2, expression of EIFA2AK3 is determined by measuring expression of a protein encoded by a sequence comprising at least 80% sequence identity to SEQ ID NO: 3 expression of GTF3C3 is determined by measuring expression of a protein encoded by a sequence comprising at least 80% sequence identity to SEQ ID NO: 4, and/or expression of LARS is determined by measuring expression of a protein encoded by a sequence comprising at least 80% sequence identity to SEQ ID NO: 5.

7. The method of any one of claims 1-6, wherein the ER+ breast cancer has low levels of Ki-67 (luminal A subtype).

8. The method of any one of claims 1-6, wherein the ER+ breast cancer has high levels of Ki-67 (luminal B subtype).

9. The method of any one of claims 1-8, wherein the ER+ breast cancer is progesterone receptor (PR) positive.

10. The method of any one of claims 1-8, wherein the ER+ breast cancer is PR negative.

11. The method of any one of claims 1-10, wherein the subject who will develop resistance to tamoxifen is one who has a recurrence of their ER+ breast cancer within one year of treatment with the tamoxifen.

12. The method of any one of claims 1-11, wherein the subject who will not develop resistance to tamoxifen is one who does not have a recurrence of their ER+ breast cancer within one year of treatment with the tamoxifen.

13. The method of any one of claims 1-12, wherein the expression comprises mRNA expression.

14. The method of any one of claims 2-13, further comprising
administering a therapeutically effective amount of tamoxifen to the subject with ER+ breast cancer who will not develop resistance to tamoxifen, thereby treating the subject with ER+ breast cancer; or
administering a therapeutically effective amount of a non-tamoxifen therapy to the subject with ER+ breast cancer who not develop resistance to tamoxifen,
thereby treating the subject.

15. The method of any one of claim 1 or 4-14, wherein the non-tamoxifen therapy comprises a therapeutically effective amount of fulvestrant, a CDK 4/6 inhibitor, a PI3K inhibitor, luteinizing-hormone releasing hormone (LHRH) agonist, aromatase inhibitor, or combinations thereof.

16. The method of any one of claim 1 or 4-15, wherein the non-tamoxifen therapy comprises a radiation therapy.

17. The method of any of one claim 1 or 4-16, wherein the treating the subject only occurs where the subject is identified as a subject who will or will not develop tamoxifen resistance with a p value of at least 0.01 or at least 0.02.

18. The method of any one of claims 2-16, wherein the subject is identified as a subject who will or will not develop tamoxifen resistance with a p value of at least 0.01 or at least 0.02.

19. The method of any one of claims 1-18, wherein the sample is an ER+ breast cancer sample.

20. The method of any one of claims 1-19, wherein the subject is a human.

21. The method of any one of claims 1-20, wherein the subject had their ER+ breast cancer surgically removed and did not yet receive tamoxifen therapy.

22. The method of any one of claims 4-21, wherein the control comprises a control risk score, and the method further comprises:
summing expression of AP2S1, expression of CDC2 multiplied by three, expression of GTF3C3, expression of EIFA2AK3, and expression of LARS multiplied by two to calculate a risk score for the ER+ breast cancer-related molecules for the sample obtained from a subject with ER+ breast cancer; and
comparing the risk score to a control risk score representing expression for the ER+ breast cancer-related molecules expected in a sample from a subject who will develop resistance to tamoxifen and/or to a control risk score representing expression for the ER+ breast cancer-related molecules expected in a sample from a subject who will not develop resistance to tamoxifen;
wherein:
the control risk score is calculated by summing expression of AP2S1, expression of CDC2 multiplied by three, expression of GTF3C3, expression of EIFA2AK3, and expression of LARS multiplied by two to calculate a risk score for the ER+ breast cancer-related molecules for samples obtained from subjects with ER+ breast cancer to form a risk score data set; calculating a mean of the risk score data set; calculating a standard deviation of the risk score data set; and summing the mean of the risk score data set with the standard deviation of the risk score data set to yield the control risk score;
wherein:
the control representing expression for the ER+ breast cancer-related molecules expected in a sample from a subject who will develop resistance to tamoxifen is values greater than the control risk score;
wherein:
the control representing expression for the ER+ breast cancer-related molecules expected in a sample from a subject who will not develop resistance to tamoxifen is values less than or equal to the control risk score.

23. The method of claim 22 wherein the control risk score is 4.5.
